(19) United States
(12) Patent Application Publication
(10) Pub. No.: US 2012/0040998 A1
(43) Pub. Date: Feb. 16, 2012

(54) 2-ALKYL PIPERIDINE MGLUR5 RECEPTOR MODULATORS

(76) Inventors: Swati P. Mercer, Philadelphia, PA (US); Anthony J. Roecker, North Wales, PA (US); Theresa Williams, Harleysville, PA (US); Xu-Fang Zhang, Dresher, PA (US)

(21) Appl. No.: 13/265,672

(22) PCT Filed: Apr. 22, 2010
(86) PCT No.: PCT/US10/32001

§ 371 Date: Oct. 21, 2011

Related U.S. Application Data
(60) Provisional application No. 61/172,012, filed on Apr. 23, 2009.
(60) Provisional application No. 61/325,579, filed on Apr. 19, 2010.

(51) Int. Cl. A61K 31/454; A61K 31/4545; A61P 25/18; C07D 417/14; C07D 413/04; A61P 25/00; C07D 413/14; A61K 31/497

(52) U.S. Cl. 514/255.05; 546/209; 514/326; 546/194; 514/318; 546/193; 544/405

(57) ABSTRACT

The present invention is directed to 2-alkylpiperidines which are positive allosteric modulators of metabotropic glutamate receptors, particularly the mGluR5 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### BACKGROUND OF THE INVENTION

The excitatory amino acid L-glutamic acid (sometimes referred to simply as L-glutamate or glutamate) through its many receptors mediates most of the excitatory neurotransmission within the mammalian central nervous system (CNS). The excitatory amino acids, including glutamate, are of great physiological importance, playing a role in a variety of physiological processes, such as long-term potentiation (learning and memory), the development of synaptic plasticity, motor control, respiration, cardiovascular regulation, and sensory perception. Glutamate acts via at least two distinct classes of receptors. One class is composed of the ionotropic glutamate (iGlu) receptors that act as ligand-gated ionic channels. Via activation of the iGlu receptors, glutamate is thought to regulate fast neuronal transmission within the synapse of two connecting neurons in the CNS. The second general type of receptor is the G-protein or second messenger-linked “metabotropic” glutamate (mGluR) receptor. Both types of receptors appear not only to mediate normal synaptic transmission along excitatory pathways, but also participate in the modification of synaptic connections during development and throughout life.

The present invention relates to modulators of metabotropic glutamate receptors, in particular subtype 5 (“mGluR5”) receptors. The mGluR receptors belong to the Type III G-protein coupled receptor (GPCR) superfamily. This superfamily of GPCR's include the calcium-sensing receptors, GABA B receptors and pheromone receptors, which are unique in that they are activated by binding of effectors to the amino-terminus portion of the receptor protein. The mGlu receptors are thought to mediate glutamate's demonstrated ability to modulate intracellular signal transduction pathways. They have been demonstrated to be localized both pre- and post-synaptically where they can regulate neurotransmitter release, either glutamate or other neurotransmitters, or modify the post-synaptic response of neurotransmitters, respectively.

At present, there are eight distinct mGlu receptors that have been positively identified, cloned, and their sequences reported. These are further subdivided into three groups (Groups I, II and III) based on their amino acid sequence homology, their ability to effect certain signal transduction mechanisms, and their known pharmacological properties. Activation of mGluRs lead to a large variety of intracellular responses and activation of different transductional cascades. Among mGluR members, the mGluR5 subtype is of high interest for counterbalancing the deficit or excesses of neurotransmission in neuropsychatric diseases. mGluR5 belongs to Group I and its activation initiates cellular responses through G-protein mediated mechanisms. mGluR5 is coupled to phospholipase C and stimulates phosphoinositide hydrolysis and intracellular calcium mobilization. In the CNS, mGluR5 receptors are abundant mainly throughout cortex, hippocampus, caudate-putamen and nucleus accumbens. As these brain areas have been shown to be involved in emotion, motivational processes and in numerous aspects of cognitive function, mGluR5 modulators are predicted to be of therapeutic interest.

It has become increasingly clear that there is a link between modulation of excitatory amino acid receptors, including the glutamatergic system, through changes in glutamate release or alteration in postsynaptic receptor activation, and a variety of neurological and psychiatric disorders. For example, a variety of potential clinical indications have been suggested to be targets for the development of subtype selective mGluR modulators. These include epilepsy, neuropathic and inflammatory pain, numerous psychiatric disorders (e.g. anxiety and schizophrenia), movement disorders (e.g. Parkinson disease), neuroprotection (stroke and head injury), migraine and addiction/drug dependency. The medical consequences of such glutamate dysfunction make the abatement of these neurological processes an important therapeutic goal.

### SUMMARY OF THE INVENTION

The present invention is directed to 2-alkylpiperidines which are positive allosteric modulators of metabotropic glutamate receptors, particularly the mGluR5 receptor, and which are useful in the treatment or prevention of neurological and psychiatric disorders associated with glutamate dysfunction and diseases in which metabotropic glutamate receptors are involved. The invention is also directed to pharmaceutical compositions comprising these compounds and the use of these compounds and compositions in the prevention or treatment of such diseases in which metabotropic glutamate receptors are involved.

### DETAILED DESCRIPTION OF THE INVENTION

The present invention is directed to compounds of the formula I:

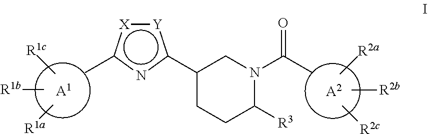

wherein:
A¹ is selected from the group consisting of phenyl, naphthyl and heteroaryl;
A² is selected from the group consisting of phenyl, naphthyl and heteroaryl;
X is selected from N, O and C(R¹³),
Y is selected from N and O, wherein X is N and Y is O, to form a oxadiazole ring, or X is O and Y is N, to form a oxadiazole ring, or X is C(R¹³) and Y is O to form an oxazole ring;
R^{1a}, R^{1b} and R^{1c} may be absent if the valency of A¹ does not permit such substitution and are independently selected from the group consisting of:
   (1) hydrogen,
   (2) halogen,
   (3) hydroxyl,
   (4) —(C═O)ₘ—Oₙ—C₁₋₆alkyl, where m is 0 or 1, n is 0 or 1 (wherein if m is 0 or n is 0, a bond is present) and where the alkyl is unsubstituted or substituted with one or more substituents selected from R¹³,
   (5) —(C═O)ₘ—Oₙ—C₁₋₆cycloalkyl, where the cycloalkyl is unsubstituted or substituted with one or more substituents selected from R¹³,
   (6) —(C═O)ₘ—C₂₋₄alkenyl, where the alkenyl is unsubstituted or substituted with one or more substituents selected from R¹³,
   (7) —(C═O)ₘ—C₂₋₄alkynyl, where the alkynyl is unsubstituted or substituted with one or more substituents selected from R¹³,
   (8) —(C═O)ₘ—Oₙ-phenyl or —(C═O)ₘ—Oₙ-napthyl, where the phenyl or naphthyl is unsubstituted or substituted with one or more substituents selected from R¹³,
   (9) —(C═O)ₘ—Oₙ-heterocycle, where the heterocycle is unsubstituted or substituted with one or more substituents selected from R¹³,
   (10) —(C═O)ₘ—NR¹⁰R¹¹, wherein R¹⁰ and R¹¹ are independently selected from the group consisting of:
      (a) hydrogen,
      (b) C₁₋₆alkyl, which is unsubstituted or substituted with R¹³,
      (c) C₃₋₆alkenyl, which is unsubstituted or substituted with R¹³,
      (d) C₃₋₆alkynyl, which is unsubstituted or substituted with R¹³,
      (e) C₃₋₆cycloalkyl which is unsubstituted or substituted with R¹³,
      (f) phenyl, which is unsubstituted or substituted with R¹³, and
      (g) heterocycle, which is unsubstituted or substituted with R¹³,
   (11) —S(O)₂—NR¹⁰R¹¹,
   (12) —S(O)_{q}—R¹², where q is 0, 1 or 2 and where R¹² is selected from the definitions of R¹⁰ and R¹¹,
   (13) —CO₂H,
   (14) —CN, and
   (15) —NO₂;
R^{2a}, R^{2b} and R^{2c} may be absent if the valency of A² does not permit such substitution and are independently selected from the group consisting of:
   (1) hydrogen,
   (2) halogen,
   (3) hydroxyl,
   (4) —(C═O)ₘ—Oₙ—C₁₋₆alkyl, where the alkyl is unsubstituted or substituted with one or more substituents selected from R¹³,
   (5) —(C═O)ₘ—Oₙ—C₃₋₆cycloalkyl, where the cycloalkyl is unsubstituted or substituted with one or more substituents selected from R¹³,
   (6) —(C═O)ₘ—C₂₋₄alkenyl, where the alkenyl is unsubstituted or substituted with one or more substituents selected from R¹³,
   (7) —(C═O)ₘ—C₂₋₄alkynyl, where the alkynyl is unsubstituted or substituted with one or more substituents selected from R¹³,
   (8) —(C═O)ₘ—Oₙ-phenyl or —(C═O)ₘ—Oₙ-napthyl, where the phenyl or naphthyl is unsubstituted or substituted with one or more substituents selected from R¹³,
   (9) —(C═O)ₘ—Oₙ-heterocycle, where the heterocycle is unsubstituted or substituted with one or more substituents selected from R¹³,
   (10) —(C═O)ₘ—NR¹⁰R¹¹,
   (11) —S(O)₂—NR¹⁰R¹¹,
   (12) —S(O)_{q}—R¹²,
   (13) —CO₂H,
   (14) —CN, and
   (15) —NO₂;
R³ is C₁₋₆alkyl;
R¹³ is selected from the group consisting of:
   (1) halogen,
   (2) hydroxyl,
   (3) —(C═O)ₘ—Oₙ—C₁₋₆alkyl, where the alkyl is unsubstituted or substituted with one or more substituents selected from R¹⁴,
   (4) —Oₙ—(C₁₋₃)perfluoroalkyl,
   (5) —(C═O)ₘ—Oₙ—C₃₋₆cycloalkyl, where the cycloalkyl is unsubstituted or substituted with one or more substituents selected from R¹⁴,
   (6) —(C═O)ₘ—C₂₋₄alkenyl, where the alkenyl is unsubstituted or substituted with one or more substituents selected from R¹⁴,
   (7) —(C═O)ₘ—C₂₋₄alkynyl, where the alkynyl is unsubstituted or substituted with one or more substituents selected from R¹⁴,
   (8) —(C═O)ₘ—Oₙ-phenyl or —(C═O)ₘ—Oₙ-napthyl, where the phenyl or naphthyl is unsubstituted or substituted with one or more substituents selected from R¹⁴,
   (9) —(C═O)ₘ—Oₙ-heterocycle, where the heterocycle is unsubstituted or substituted with one or more substituents selected from R¹⁴,
   (10) —(C═O)ₘ—NR¹⁰R¹¹,
   (11) —S(O)₂—NR¹⁰R¹¹,
   (12) —S(O)_{q}—R¹²,
   (13) —CO₂H,
   (14) —CN, and
   (15) —NO₂;
R¹⁴ is selected from the group consisting of:
   (1) hydroxyl,
   (2) halogen,
   (3) C₁₋₆alkyl,
   (4) —C₃₋₆cycloalkyl,
   (5) —O—C₁₋₆alkyl,
   (6) —O(C═O)—C₁₋₆alkyl,
   (7) —NH—C₁₋₆alkyl,
   (8) phenyl,
   (9) heterocycle,
   (10) —CO₂H, and
   (11) —CN;
      or a pharmaceutically acceptable salt thereof.

An embodiment of the present invention includes compounds of the formula I′:

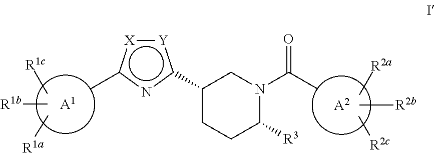

wherein A¹, A², X, Y, R^{1a}, R^{1b}, R^{1c}, R^{2a}, R^{2b}, R^{2c} and R³ are defined herein; or a pharmaceutically acceptable salt thereof.

An embodiment of the present invention includes compounds of the formula Ia:

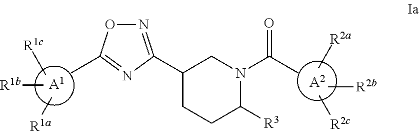

wherein A¹, A², R^{1a}, R^{1b}, R^{1c}, R^{2a}, R^{2b}, R^{2c} and R³ are defined herein; or a pharmaceutically acceptable salt thereof.

An embodiment of the present invention includes compounds of the formula Ia′:

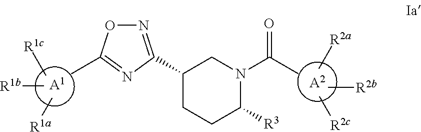

wherein A¹, A², R^{1a}, R^{1b}, R^{1c}, R^{2a}, R^{2b}, R^{2c} and R³ are defined herein; or a pharmaceutically acceptable salt thereof.

An embodiment of the present invention includes compounds of the formula Ib:

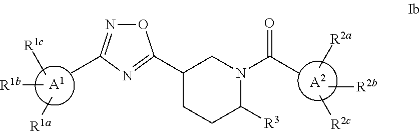

wherein A¹, A², R^{1a}, R^{1b}, R^{1c}, R^{2a}, R^{2b}, R^{2c} and R³ are defined herein; or a pharmaceutically acceptable salt thereof.

An embodiment of the present invention includes compounds of the formula Ib′:

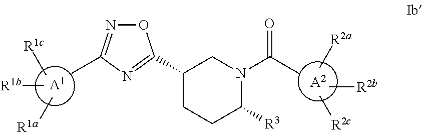

wherein A¹, A², R^{1a}, R^{1b}, R^{1c}, R^{2a}, R^{2b}, R^{2c} and R³ are defined herein; or a pharmaceutically acceptable salt thereof.

An embodiment of the present invention includes compounds of the formula Ic:

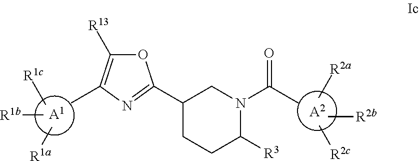

wherein A¹, A², R^{1a}, R^{1b}, R^{1c}, R^{2a}, R^{2b}, R^{2c}, R³ and R¹³ are defined herein; or a pharmaceutically acceptable salt thereof.

An embodiment of the present invention includes compounds of the formula Ic′:

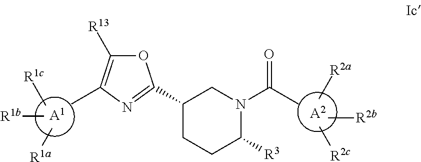

wherein A¹, A², R^{1a}, R^{1b}, R^{1c}, R^{2a}, R^{2b}, R^{2c}, R³ and R¹³ are defined herein; or a pharmaceutically acceptable salt thereof.

An embodiment of the present invention includes compounds of the formula Id:

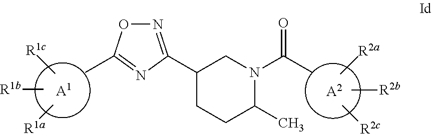

wherein A¹, A², R^{1a}, R^{1b}, R^{1c}, R^{2a}, R^{2b} and R^{2c} are defined herein; or a pharmaceutically acceptable salt thereof.

An embodiment of the present invention includes compounds of the formula Id′:

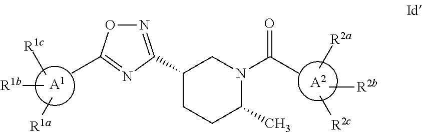

wherein A¹, A², R^{1a}, R^{1b}, R^{1c}, R^{2a}, R^{2b} and R^{2c} are defined herein; or a pharmaceutically acceptable salt thereof.

An embodiment of the present invention includes compounds of the formula Ie:

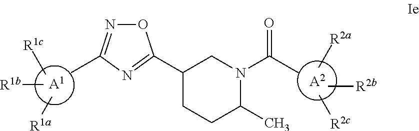

wherein A¹, A², R^{1a}, R^{1b}, R^{1c}, R^{2a}, R^{2b} and R^{2c} are defined herein; or a pharmaceutically acceptable salt thereof.

An embodiment of the present invention includes compounds of the formula Ie′:

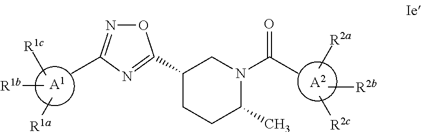

wherein A¹, A², R^{1a}, R^{1b}, R^{1c}, R^{2a}, R^{2b} and R^{2c} are defined herein; or a pharmaceutically acceptable salt thereof.

An embodiment of the present invention includes compounds of the formula If:

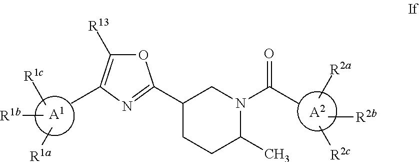

wherein A¹, A², R^{1a}, R^{1b}, R^{1c}, R^{2a}, R^{2b}, R^{2c} and R¹³ are defined herein; or a pharmaceutically acceptable salt thereof.

An embodiment of the present invention includes compounds of the formula If′:

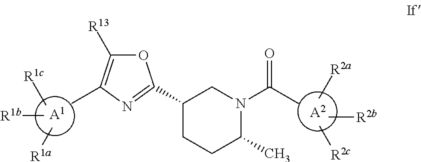

wherein A¹, A², R^{1a}, R^{1b}, R^{1c}, R^{2a}, R^{2b}, R^{2c} and R¹³ are defined herein; or a pharmaceutically acceptable salt thereof.

An embodiment of the present invention includes compounds wherein A¹ is selected from the group consisting of phenyl, pyridyl and pyrrolyl. An embodiment of the present invention includes compounds wherein A¹ is phenyl. An embodiment of the present invention includes compounds wherein A¹ is heteroaryl. An embodiment of the present invention includes compounds wherein A¹ is pyridyl. An embodiment of the present invention includes compounds wherein A¹ is pyrrolyl.

An embodiment of the present invention includes compounds wherein A² is selected from the group consisting of: phenyl and pyridyl. An embodiment of the present invention includes compounds where A² is phenyl. An embodiment of the present invention includes compounds wherein A² is heteroaryl. An embodiment of the present invention includes compounds wherein A² is pyridyl.

An embodiment of the present invention includes compounds wherein X is N and Y is O, to form a oxadiazole ring. An embodiment of the present invention includes compounds wherein X is O and Y is N, to form a oxadiazole ring. An embodiment of the present invention includes compounds wherein X is C(R¹³) and Y is O to form an oxazole ring.

An embodiment of the present invention includes compounds wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl, phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, —O—C₁₋₆alkyl or —NO₂.

An embodiment of the present invention includes compounds wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl, and
(5) —O—C₁₋₆alkyl, which is unsubstituted or substituted with halogen, hydroxyl or phenyl.

An embodiment of the present invention includes compounds wherein R^{1a}, R^{1b} and R^{1c} are independently selected from the group consisting of:
(1) hydrogen,
(2) halogen, and
(3) C₁₋₆alkyl.

An embodiment of the present invention includes compounds wherein R^{1a}, R^{1b} and R^{1c} are independently selected from the group consisting of:
(1) hydrogen,
(2) chloro,
(3) fluroro, and
(4) methyl.

An embodiment of the present invention includes compounds wherein A¹ is phenyl, pyridyl or pyrrolyl and R^{1a}, R^{1b} and R^{1c} are independently selected from the group consisting of:
(1) hydrogen,
(2) chloro,
(3) fluroro, and
(4) methyl.

An embodiment of the present invention includes compounds wherein A¹ is phenyl and wherein R^{1a} is halogen, R^{1b} is hydrogen and R^{1c} is hydrogen. An embodiment of the present invention includes compounds wherein A¹ is phenyl and wherein R^{1a} is fluoro, R^{1b} is hydrogen and R^{1c} is hydrogen. An embodiment of the present invention includes compounds wherein A¹ is phenyl and wherein R^{1a} is chloro, R^{1b} is hydrogen and R^{1c} is hydrogen. An embodiment of the present invention includes compounds wherein A¹ is phenyl and wherein R^{1a} is methyl, R^{1b} is hydrogen and R^{1c} is hydrogen.

An embodiment of the present invention includes compounds wherein A¹ is pyridyl and wherein R^{1a} is halogen, R^{1b} is hydrogen and R^{1c} is hydrogen. An embodiment of the present invention includes compounds wherein A¹ is pyridyl and wherein R^{1a} is fluoro, R^{1b} is hydrogen and R^{1c} is hydrogen. An embodiment of the present invention includes compounds wherein A¹ is pyridyl and wherein R^{1a} is chloro, R^{1b} is hydrogen and R^{1c} is hydrogen. An embodiment of the present invention includes compounds wherein A¹ is pyridyl and wherein R^{1a} is methyl, R^{1b} is hydrogen and R^{1c} is hydrogen.

An embodiment of the present invention includes compounds wherein A¹ is pyrrolyl and wherein R^{1a} is halogen, R^{1b} is hydrogen and R^{1c} is hydrogen. An embodiment of the present invention includes compounds wherein A¹ is pyrrolyl and wherein R^{1a} is fluoro, R^{1b} is hydrogen and R^{1c} is hydrogen. An embodiment of the present invention includes compounds wherein A¹ is pyrrolyl and wherein R^{1a} is chloro, R^{1b} is hydrogen and R^{1c} is hydrogen. An embodiment of the present invention includes compounds wherein A¹ is pyrrolyl and wherein R^{1a} is methyl, R^{1b} is hydrogen and R^{1c} is hydrogen.

An embodiment of the present invention includes compounds wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, —O—C₁₋₆alkyl or —NO₂.

An embodiment of the present invention includes compounds wherein R^{2a}, R^{2b} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl,
(5) —O—C₁₋₆alkyl, which is unsubstituted or substituted with halogen, hydroxyl or phenyl, and
(6) —NH—C₁₋₆alkyl, or —N(C₁₋₆alkyl)(C₁₋₆alkyl), which is unsubstituted or substituted with halogen.

An embodiment of the present invention includes compounds wherein R^{2a}, R^{2b} and R^{2c} are independently selected from the group consisting of:
(1) hydrogen,
(2) halogen,
(3) C₁₋₆alkyl, which is unsubstituted or substituted with halogen,
(4) —O—C₁₋₆alkyl, which is unsubstituted or substituted with halogen, and
(5) —NH—C₁₋₆alkyl, or —N(C₁₋₆alkyl)(C₁₋₆alkyl), which is unsubstituted or substituted with halogen.

An embodiment of the present invention includes compounds wherein R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

An embodiment of the present invention includes compounds wherein R^{2a}, R^{2b} and R^{2c} are independently selected from the group consisting of:
(1) hydrogen,
(2) chloro,
(3) fluoro, and
(4) methoxy.

An embodiment of the present invention includes compounds wherein A² is phenyl or pyridyl and R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

An embodiment of the present invention includes compounds wherein A² is phenyl or pyridyl and R^{2a}, R^{2b} and R^{2c} are independently selected from the group consisting of:
(1) hydrogen,
(2) chloro,
(3) fluoro, and
(4) methoxy.

An embodiment of the present invention includes compounds wherein A² is phenyl and wherein R^{2a} is halogen or methoxy, R^{2b} is hydrogen and R^{2c} is hydrogen. An embodiment of the present invention includes compounds wherein A² is phenyl and wherein R^{2a} is fluoro, R^{2b} is hydrogen and R^{2c} is hydrogen. An embodiment of the present invention includes compounds wherein A² is phenyl and wherein R^{2a} is chloro, R^{2b} is hydrogen and R^{2c} is hydrogen. An embodiment of the present invention includes compounds wherein A² is phenyl and wherein R^{2a} is methoxy, R^{2b} is hydrogen and R^{2c} is hydrogen. An embodiment of the present invention includes compounds wherein A² is phenyl and wherein R^{2a} is fluroro, R^{2b} is fluoro and R^{2c} is hydrogen. An embodiment of the present invention includes compounds wherein A² is phenyl and wherein R^{2a} is fluoro, R^{2b} is methoxy and R^{2c} is hydrogen. An embodiment of the present invention includes compounds wherein A² is phenyl and wherein R^{2a} is methoxy, R^{2b} is methoxy and R^{2c} is hydrogen.

An embodiment of the present invention includes compounds wherein A² is pyridyl and wherein R^{2a} is halogen or methoxy, R^{2b} is hydrogen and R^{2c} is hydrogen. An embodiment of the present invention includes compounds wherein A² is pyridyl and wherein R^{2a} is fluoro, R^{2b} is hydrogen and R^{2c} is hydrogen. An embodiment of the present invention includes compounds wherein A² is pyridyl and wherein R^{2a} is chloro, R^{2b} is hydrogen and R^{2c} is hydrogen. An embodiment of the present invention includes compounds wherein A² is pyridyl and wherein R^{2a} is methoxy, R^{2b} is hydrogen and R^{2c} is hydrogen. An embodiment of the present invention includes compounds wherein A² is pyridyl and wherein R^{2a} is fluroro, R^{2b} is fluoro and R^{2c} is hydrogen. An embodiment of the present invention includes compounds wherein A² is pyridyl and wherein R^{2a} is fluoro, R^{2b} is methoxy and R^{2c} is hydrogen. An embodiment of the present invention includes compounds wherein A² is pyridyl and wherein R^{2a} is methoxy, R^{2b} is methoxy and R^{2c} is hydrogen.

An embodiment of the present invention includes compounds wherein R³ is C₁₋₄alkyl. An embodiment of the present invention includes compounds wherein R³ is methyl. An embodiment of the present invention includes compounds wherein R³ is ethyl.

An embodiment of the present invention includes compounds wherein R³ is in the cis-orientation on the piperidine ring relative to the substituent bearing the oxadiazole ring.

An embodiment of the present invention includes compounds wherein is R¹³ hydrogen.

Specific embodiments of the present invention include a compound which is selected from the group consisting of the subject compounds of the Examples herein and pharmaceutically acceptable salts thereof and individual enantiomers and diastereomers thereof.

As appreciated by those of skill in the art, halogen or halo as used herein are intended to include luorine, chlorine, bromine and iodine. Similarly, “alkyl”, as well as other groups having the prefix “alk”, such as alkoxy, alkanoyl, means carbon chains which may be linear or branched or combinations thereof. C₁₋₆, as in C₁₋₆alkyl is defined to identify the group as having 1, 2, 3, 4, 5 or 6 carbons in a linear or branched arrangement, such as methyl, ethyl, n-propyl, iso-propyl, n-butyl, iso-butyl, sec-butyl, tert-butyl, pentyl, hexyl, and the like. “Alkylene” means a straight or branched chain of carbon atoms with a group substituted at both ends, such as —CH₂CH₂— and —CH₂CH₂CH₂—. “Alkenyl” means carbon chains which contain at least one carbon-carbon double bond, and which may be linear or branched or combinations thereof such that C₂₋₆alkenyl is defined to identify the group as having 2, 3, 4, 5 or 6 carbons which incorporates at least one double bond, which may be in a E- or a Z-arrangement, including vinyl, allyl, isopropenyl, pentenyl, hexenyl, heptenyl, 1-propenyl, 2-butenyl, 2-methyl-2-butenyl, and the like. “Alkynyl” means carbon chains which contain at least one carbon-carbon triple bond, and which may be linear or branched or combinations thereof, such as ethynyl, propargyl, 3-methyl-1-pentynyl, 2-heptynyl and the like. “Cycloalkyl” means mono-, bi- or tri-cyclic structures, optionally combined with linear or branched structures, having the indicated number of carbon atoms, such as cyclopropyl, cyclopentyl, cycloheptyl, adamantyl, cyclododecylmethyl, 2-ethyl-1-bicyclo[4.4.0]decyl, and the like. “Alkoxy” means alkoxy groups of a straight or branched having the indicated number of carbon atoms. C₁₋₆alkoxy, for example, includes methoxy, ethoxy, propoxy, isopropoxy, and the like. “Heteroaryl” means mono- or bicyclic aromatic rings with at least one ring containing a heteroatom selected from N, O and S, and each ring containing 5 or 6 atoms. Examples of heteroaryl include benzoimidazolyl, benzimidazolonyl, benzofuranyl, benzofurazanyl, benzopyrazolyl, benzothiazolyl, benzotriazolyl, benzothiophenyl, benzoxazepin, benzoxazolyl, carbazolyl, carbolinyl, cinnolinyl, furanyl, furo(2,3-b)pyridyl, imidazolyl, indolinyl, indolyl, dihydroindolyl, indolazinyl, indazolyl, isobenzofuranyl, isoindolyl, isoquinolyl, isothiazolyl, isoxazolyl, naphthpyridinyl, oxadiazolyl, oxazolyl, oxazoline, isoxazoline, oxetanyl, pyrazinyl, pyrazolyl, pyridazinyl, pyridopyridinyl, pyridazinyl, pyridyl, pyrimidyl, pyrrolyl, quinazolinyl, quinolyl, quinoxalinyl, tetrahydroquinoxalinyl, tetrazolyl, tetrazolopyridyl, thiadiazolyl, thiazolyl, thienyl, triazolyl, and N-oxides thereof, and the like.

A group which is designated as being independently substituted with substituents may be independently substituted with multiple numbers of such substituents.

The compounds of the present invention may contain one or more asymmetric centers and can thus occur as racemates and racemic mixtures, single enantiomers, diastereomeric mixtures and individual diastereomers. Additional asymmetric centers may be present depending upon the nature of the various substituents on the molecule. Each such asymmetric center will independently produce two optical isomers and it is intended that all of the possible optical isomers and diastereomers in mixtures and as pure or partially purified compounds are included within the ambit of this invention. Any formulas, structures or names of compounds described in this specification that do not specify a particular stereochemistry are meant to encompass any and all existing isomers as described above and mixtures thereof in any proportion. When stereochemistry is specified, the invention is meant to encompass that particular isomer in pure form or as part of a mixture with other isomers in any proportion.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration. If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art. Alternatively, any enantiomer of a compound may be obtained by stereoselective synthesis using optically pure starting materials or reagents of known configuration by methods well known in the art.

The present invention also includes all pharmaceutically acceptable isotopic variations of a compound of the Formula I in which one or more atoms is replaced by atoms having the same atomic number, but an atomic mass or mass number different from the atomic mass or mass number usually found in nature. Examples of isotopes suitable for inclusion in the compounds of the invention include isotopes of hydrogen such as 2H and 3H, carbon such as ¹¹C, ¹³C and ¹⁴C, nitrogen such as ¹³N and ¹⁵N, oxygen such as ¹⁵O, ¹⁷O and ¹⁸O, phosphorus such as ³²P, sulfur such as ³⁵S, fluorine such as ¹⁸F, iodine such as ²³I and ¹²⁵I, and chlorine such as ³⁶Cl. Certain isotopically-labelled compounds of Formula I, for example those incorporating a radioactive isotope, are useful in drug and/or substrate tissue distribution studies. The radioactive isotopes tritium, i.e. ³H, and carbon-14, i.e. ¹⁴C, are particularly useful for this purpose in view of their ease of incorporation and ready means of detection. Substitution with heavier isotopes such as deuterium, i.e. ²H, may afford certain therapeutic advantages resulting from greater metabolic stability, for example, increased in vivo half-life or reduced dosage requirements, and hence may be preferred in some circumstances. Substitution with positron emitting isotopes, such as ¹¹C, ¹⁸F, ¹⁵O and ¹³N, can be useful in Positron Emission Topography (PET) studies for examining substrate receptor occupancy. Isotopically-labelled compounds of Formula I can generally be prepared by conventional techniques known to those skilled in the art or by processes analogous to those described in the accompanying Examples using appropriate isotopically-labelled reagents in place of the non-labelled reagent previously employed.

The term “pharmaceutically acceptable salts” refers to salts prepared from pharmaceutically acceptable non-toxic bases or acids including inorganic or organic bases and inorganic or organic acids. Salts derived from inorganic bases include aluminum, ammonium, calcium, copper, ferric, ferrous, lithium, magnesium, manganic salts, manganous, potassium, sodium, zinc, and the like. Particular embodiments include the ammonium, calcium, magnesium, potassium, and sodium salts. Salts in the solid form may exist in more than one crystal structure, and may also be in the form of hydrates. Salts derived from pharmaceutically acceptable organic non-toxic bases include salts of primary, secondary, and tertiary amines, substituted amines including naturally occurring substituted amines, cyclic amines, and basic ion exchange resins, such as arginine, betaine, caffeine, choline, N,N′-dibenzylethylene-diamine, diethylamine, 2-diethylaminoethanol, 2-dimethylamino-ethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine, and the like. When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particular embodiments citric, hydrobromic, hydrochloric, maleic, phosphoric, sulfuric, fumaric, and tartaric acids. It will be understood that, as used herein, references to the compounds of the present invention are meant to also include the pharmaceutically acceptable salts.

Exemplifying the invention are the specific compounds disclosed in the Examples and herein. The subject compounds are useful in a method of enhancing the neuromodulatory effect of metabotorpic glutamate receptor activity in a patient such as a mammal in need of such enhancement comprising the administration of an effective amount of the compound. The present invention is directed to the use of the subject compounds disclosed herein as positive allosteric modulators of metabotropic glutamate receptor activity.

The invention also encompasses a pharmaceutical composition comprising a compound of Formula I in combination with a pharmaceutically acceptable carrier.

The invention also encompasses a method for treating a neurological or psychiatric disorder associated with glutamate dysfunction in a patient in need thereof comprising administering to the patient a therapeutically effective amount of a compound of Formula I. The invention also encompasses this method wherein the neurological or psychiatric disorder associated with glutamate dysfunction is schizophrenia.

The compounds of the present invention are modulators of metabotropic glutamate (mGluR) receptor function, in particular they are positive allosteric modulators of mGluR5 receptors. That is, the compounds of Formula I do not appear to bind to the orthosteric glutamate recognition site, and do not activate the mGluR5 by themselves. Instead, the response of mGluR5 to a concentration of glutamate or mGluR5 agonist is increased when a compound of Formula I is present. The compounds of Formula I are expected to have their effect at mGluR5 by virtue of their ability to enhance the function of the receptor. It is recognized that the compounds of the present invention would be expected to increase the effectiveness of glutamate and glutamate agonists of the mGluR5 receptor. Thus, the compounds of the present invention are expected to be useful in the treatment of various neurological and psychiatric disorders associated with glutamate dysfunction described to be treated herein and others that can be treated by such positive allosteric modulators as are appreciated by those skilled in the art.

The present invention is directed to the use of the compounds disclosed herein as positive allosteric modulators of mGluR5 receptor activity. The present invention is directed to a compound of the present invention or a pharmaceutically acceptable salt thereof for use in medicine. The present invention is further directed to a use of a compound of the present invention or a pharmaceutically acceptable salt thereof for the manufacture of a medicament for positive allosteric modulatorion of mGluR5 receptor activity or treating the disorders and diseases noted herein in humans and animals.

The present invention is further directed to a method for the manufacture of a medicament for positive allosteric modulation of metabotropic glutamate receptor activity in humans and animals comprising combining a compound of the present invention with a pharmaceutical carrier or diluent.

The subject treated in the present methods is generally a mammal, preferably a human being, male or female, in whom potentiation of metabotropic glutamate receptor activity is desired. In addition to primates, especially humans, a variety of other mammals can be treated according to the method of the present invention. The term “therapeutically effective amount” means the amount of the subject compound that will elicit the biological or medical response of a tissue, system, animal or human that is being sought by the researcher, veterinarian, medical doctor or other clinician. It is recognized that one skilled in the art may affect the neurological and psychiatric disorders by treating a patient presently afflicted with the disorders or by prophylactically treating a patient afflicted with the disorders with an effective amount of the compound of the present invention. As used herein, the terms “treatment” and “treating” refer to all processes wherein there may be a slowing, interrupting, arresting, controlling, or stopping of the progression of the neurological and psychiatric disorders described herein, but does not necessarily indicate a total elimination of all disorder symptoms, as well as the prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.

The utility of the compounds in accordance with the present invention as positive allosteric modulators of metabotropic glutamate receptor activity, in particular mGluR5 activity, may be readily determined without undue experimentation by methodology well known in the art, including O'Brien et al., Molecular Pharmacology 2003, 64(3) 731-740. In particular, the compounds of the following examples had activity in reference assays by enhancing mGluR5 activity. The utility of the compounds as modulators of metabotropic glutamate receptor 5 (mGluR5) activation was demonstrated by their ability to increase an intracellular calcium flux above that achieved by a sub-threshold level of natural agonist (glutamate). Changes in intracellular Ca²⁺ were measured with Fluo-4AM ester (Invitrogen/Molecular Probes), which was detected on a Fluorometric Imaging Plate Reader (FLIPR, Molecular Devices, Sunnyvale, Calif.). In a typical experiment the mGluR5 positive allosteric modulatory activity of the compounds of the present invention was determined in accordance with the following experimental method.

Cell Culture: Chinese Hamster Ovary (CHO) cells expressing human mGluR5_{A} were maintained in growth medium containing DMEM, 10% dialyzed Fetal Bovine Serum, 50 units/mL Penicillin, 50 ug/mL Streptomycin, 2 mM L-glutamine, 1×MEM non-essential amino acids, 1 mM sodium pyruvate, 25 mM HEPES, 55 uM 2-mercaptoethanol, 5 ug/mL Puromycin, and 250 ug/mL Zeocin at 37° C. and 5% CO₂. The day before the experiment, the cells were washed and seeded in “plating media” containing only DMEM, 10% dialyzed Fetal Bovine Serum, 50 units/mL Penicillin, and 50 ug/mL Streptomycin at a density of 50,000 cells/well (100 uL/well) in black 384-well clear-bottom PDL-coated plates. The cells were grown overnight at 37° C. and 6% CO₂. This overnight glutamine/glutamate starvation allowed for consistent expression of the mGluR5_{A} receptor, and the ability to add a known amount of agonist (glutamate in most cases) on the day of the experiment.

Fluorescent Ca²⁺ mobilization (FLIPR) assay: The day of the experiment, the cells were washed with 37° C. Assay Buffer (Hanks Balanced Salt Solution with CaCl₂ and MgCl₂, 20 mM HEPES, 2.5 mM Probenecid, 0.1% BSA) with an automated plate washer (3×100 uL, aspiration 3 mm from bottom leaving ˜30 uL of buffer in each well). After washing, 30 uL of dye loading buffer (4 uM Fluo-4AM, 0.04% Pluronic acid, and 1% dialyzed FBS in assay buffer) were added to each well of the plates for 2 uM Fluo-4AM final concentration. The plates were incubated at 37° C. and 6% CO₂ for 1 hour to allow for dye loading. After dye loading, the cells were washed again as above, and placed on the FLIPR. Assays were conducted with two possible scenarios: 1) To determine the potencies of the compounds, as either agonists of mGluR5 or potentiators of mGluR5 in the presence of a sub-threshold amount of glutamate, 10-point titrations of the compounds (1:3 dilution between each point, 30-0.0015 uM final concentrations) were added to the cells, followed by the addition of the EC₂₀ of glutamate (300 nM) to the cells. 2) To determine the cooperativity of the compounds with the natural agonist (glutamate), single concentrations of the compounds were added to the cells, followed by the addition of a 10-point titration of glutamate (1:3 dilution between each point, 1000-0.05 uM final concentrations). When compared to the EC₅₀ of glutamate in the presence of DMSO only on the same assay plate, a left-shift in the glutamate dose-response curve in the presence of compound demonstrates the degree of potentiation at the single concentration of the compound. For both scenarios above, operation of the FLIPR was the same. Baseline fluorescence was monitored for 10 seconds, followed by the addition of compounds diluted in Assay Buffer (1% DMSO concentration after this addition, 0.66% final DMSO concentration after agonist addition). After monitoring fluorescence for 5 minutes, during which time any intrinsic agonist activity of the compounds would have been detected, the agonist (glutamate) also diluted in assay buffer was then added to the cells. The response was then monitored for an additional 3 minutes. In scenario #1, the peak during the final 3 minutes was used for potentiator data, and the peak during the 5 minutes post compound addition was used for compound agonist data. Inflection points for potentiation and agonism were determined with non-linear curve fitting, and the maximal response of the compound was compared to the maximal response of the agonist (1 mM glutamate) to provide a % of max activity for each compound. Additionally, the maximal response of each compound was compared to the sub-threshold response of the agonist (300 nM glutamate) to provide a fold potentiation value at the maximal response.

Potencies for the compounds are reported as EC₅₀ values for agonism (in the absence of 300 nM glutamate) “EC₅₀ values” (actually inflection points) for potentiation (in the presence of 300 nM glutamate).

$$\% Max\_ 1 mM glutamate = \frac{fluorescence counts caused by compound}{fluorescence counts caused by 1 mM glutamate} \times 100 \%$$

$$fold potentiation = \frac{fluorescence counts caused by compound}{fluorescence counts caused by 300 nM glutamate}$$

In scenario #2, the peak during the final 3 minutes was used for the points of the agonist dose response curve. The EC₅₀ values for the agonist in the presence of 0.66% DMSO or each single concentration of the compound were determined with non-linear curve fitting. By dividing the EC₅₀ of glutamate+DMSO by the EC₅₀ of glutamate+compound, the resulting value is the fold-shift in agonist potency, and therefore the degree of potentiation of the compound at the given concentration. This value is called the “glutamate shift”

$$Glutamate shift = \frac{{EC}_{50} of glutamate in the presence of 0.66 \% DMSO}{\begin{matrix}{EC}_{50} of glutamate in the presence \\ of a given concentration of compound\end{matrix}}$$

The compounds of the following Examples were tested and had activity as positive allosteric modulators of the mGluR5 receptor in the foregoing assays. In particular, the compounds of the following Examples had activity in potentiating the mGluR5 receptor in the FLIPR assay with an EC₅₀ of less than about 10 μM. The compounds of Examples 1-16, 2-3, 2-4, 4-4, 5-5, 9-9, 9-18, and 9-22 were tested and demonstrated activity in enhancing the mGluR5 receptor in the FLIPR assay, generally with an EC₅₀ of less than about 1 μM. The compounds of Examples 1-16, 2-3, 2-4, 4-4, 5-5, 9-9, 9-18, and 9-22 exhibited a glutamate shift of at least 7× at 10 μM. Such results are indicative of the intrinsic activity of the compounds for use as potentiators of mGluR5 receptor activity. For a compound to have therapeutic utility, it is expected that such compound should have activity in enhancing the mGluR5 receptor in the FLIPR assay with an EC₅₀ of less than about 10 μM.

TABLE 1
Representative FLIPR EC₅₀ Values

| Example | EC50 | |
|---|---|---|
| 1-16 | 124 | nM |
| 2-3 | 486 | nM |
| 2-4 | 25 | nM |
| 4-4 | 210 | nM |
| 5-5 | 19 | nM |
| 9-9 | 146 | nM |
| 9-18 | 91 | nM |
| 9-22 | 55 | nM |

With respect to other piperidinyl compounds, such as those described in PCT Patent Publications WO 2005/044797, WO 2006/123249, WO 2006/123255, WO 2006/123257 and WO 2008/056259, the present compounds exhibit unexpected properties, such as increased oral bioavailability, longer half-life, increased metabolic stability and/or maintained potency. For example, the present compounds exhibited relatively longer half-life upon oral administration to rats and dogs.

Metabotropic glutamate receptors including the mGluR5 receptor have been implicated in a wide range of biological functions. This has suggested a potential role for these receptors in a variety of disease processes in humans or other species. See e.g., Byrnes, et al., Neurotherapeutics, 6, 94-107 (2009). The compounds of the present invention have utility in treating, preventing, ameliorating, controlling or reducing the risk of a variety of neurological and psychiatric disorders associated with glutamate dysfunction, including one or more of the following conditions or diseases: schizophrenia or psychosis including schizophrenia (paranoid, disorganized, catatonic, undifferentiated, or residual type), schizophreniform disorder, schizoaffective disorder, for example of the delusional type or the depressive type, delusional disorder, psychotic disorder, brief psychotic disorder, shared psychotic disorder, psychotic disorder due to a general medical condition and substance-induced or drug-induced (for example psychosis induced by alcohol, amphetamine, cannabis, cocaine, hallucinogens, inhalants, opioids, phencyclidine, ketamine and other dissociative anaesthetics, and other psychostimulants), psychosispsychotic disorder, psychosis associated with affective disorders, brief reactive psychosis, schizoaffective psychosis, “schizophrenia-spectrum” disorders such as schizoid or schizotypal personality disorders, personality disorder of the paranoid type, personality disorder of the schizoid type, illness associated with psychosis (such as major depression, manic depressive (bipolar) disorder, Alzheimer's disease and post-traumatic stress syndrome), including both the positive and the negative symptoms of schizophrenia and other psychoses; disorders that comprise as a symptom a deficiency in attention and/or cognition; cognitive disorders including dementia (associated with Alzheimer's disease, ischemia, multi-infarct dementia, trauma, intracranial tumors, cerebral trauma, vascular problems or stroke, alcoholic dementia or other drug-related dementia, AIDS, HIV disease, Parkinson's disease, Huntington's disease, Pick's disease, Creutzfeldt-Jacob disease, perinatal hypoxia, other general medical conditions or substance abuse); Alzheimer's disease, multi-infarct dementia, AIDS-related dementia, and Fronto temperal dementia; delirium, amnestic disorders or age related cognitive decline; migraine, migraine headache; pain including acute pain, chronic pain, severe pain, intractable pain, neuropathic pain, post-traumatic pain, bone and joint pain (osteoarthritis), repetitive motion pain, dental pain, cancer pain, myofascial pain (muscular injury, fibromyalgia), perioperative pain (general surgery, gynecological), chronic pain, neuropathic pain; trigeminal neuralgia; amyotrophic lateral sclerosis (ALS); cerebral deficits subsequent to cardiac bypass surgery and grafting, stroke, cerebral ischemia, spinal cord trauma, head trauma, perinatal hypoxia, cardiac arrest, hypoglycemic neuronal damage; spinal cord injury; neuronal regeneration; neuronal inflammation; anxiety disorders including acute stress disorder, agoraphobia, generalized anxiety disorder, obsessive-compulsive disorder, panic attack, panic disorder, post-traumatic stress disorder, separation anxiety disorder, social phobia, specific phobia, substance-induced anxiety disorder and anxiety due to a general medical condition; substance-related disorders and addictive behaviors (including substance-induced delirium, persisting dementia, persisting amnestic disorder, psychotic disorder or anxiety disorder, drug addiction, tolerance, dependence or withdrawal from substances including alcohol, amphetamines, cannabis, cocaine, hallucinogens, inhalants, nicotine, opioids, phencyclidine, sedatives, hypnotics or anxiolytics); obesity, bulimia nervosa and compulsive eating disorders; bipolar disorders, mood disorders including depressive disorders, major depressive episode of the mild, moderate or severe type, a manic or mixed mood episode, a hypomanic mood episode, a depressive episode with atypical features, a depressive episode with melancholic features, a depressive episode with catatonic features, a mood episode with postpartum onset, post-stroke depression; major depressive disorder, dysthymic disorder, minor depressive disorder, premenstrual dysphoric disorder, post-psychotic depressive disorder of schizophrenia, a major depressive disorder superimposed on a psychotic disorder such as delusional disorder or schizophrenia, a bipolar disorder, for example bipolar I disorder, bipolar II disorder, cyclothymic disorder, depression including unipolar depression, seasonal depression and post-partum depression, premenstrual syndrome (PMS) and premenstrual dysphoric disorder (PDD), mood disorders due to a general medical condition, and substance-induced mood disorders; learning disorders, for example reading disorder, mathematics disorder, or a disorder of written expression, attention-deficit/hyperactivity disorder, and age-related cognitive decline, pervasive developmental disorder including autistic disorder, attention disorders including attention-deficit hyperactivity disorder (ADHD) and conduct disorder; NMDA receptor-related disorders such as autism, depression, benign forgetfulness, childhood learning disorders and closed head injury; neurodegenerative disorders or conditions, neurodegeneration associated with cerebral trauma; neurodegeneration associated with stroke, neurodegeneration associated with cerebral infarct, hypoglycemia-induced neurodegeneration, neurodegeneration associated with epileptic seizure, neurodegeneration associated with neurotoxin poisoning, multi-system atrophy; movement disorders, including akinesias and akinetic-rigid syndromes (including Parkinson's disease, drug-induced parkinsonism, postencephalitic parkinsonism, progressive supranuclear palsy, multiple system atrophy, corticobasal degeneration, parkinsonism-ALS dementia complex and basal ganglia calcification), medication-induced parkinsonism (such as neuroleptic-induced parkinsonism, neuroleptic malignant syndrome, neuroleptic-induced acute dystonia, neuroleptic-induced acute akathisia, neuroleptic-induced tardive dyskinesia and medication-induced postural tremor), Huntington's disease, dyskinesia associated with dopamine agonist therapy, Gilles de la Tourette's syndrome, epilepsy, muscular spasms and disorders associated with muscular spasticity or weakness including tremors; dyskinesias,including tremor (such as rest tremor, postural tremor, intention tremor and essential tremor), tardive dyskinesia, restless leg syndrome, chorea (such as Sydenham's chorea, Huntington's disease, benign hereditary chorea, neuroacanthocytosis, symptomatic chorea, drug-induced chorea and hemiballism), myoclonus (including generalised myoclonus and focal myoclonus), tics (including simple tics, complex tics and symptomatic tics), dystonia (including generalised dystonia such as iodiopathic dystonia, drug-induced dystonia, symptomatic dystonia and paroxymal dystonia, and focal dystonia such as blepharospasm, oromandibular dystonia, spasmodic dysphonia, spasmodic torticollis, axial dystonia, dystonic writer's cramp and hemiplegic dystonia)]; urinary incontinence; neuronal damage including ocular damage, retinopathy or macular degeneration of the eye, tinnitus, hearing impairment and loss, and brain edema; emesis; and sleep disorders including insomnia and narcolepsy.

Among the disorders above, of particular importance are the treatment of schizophrenia, migraine, anxiety (including agoraphobia, generalized anxiety disorder (GAD), obsessive-compulsive disorder (OCD), panic disorder, posttraumatic stress disorder (PTSD), social phobia, other phobias, substance-induced anxiety disorder), mood disorders (including bipolar disorders (I & II), cyclothymic disorder, depression, dysthymic disorder, major depressive disorder, substance-induced mood disorder), attention-deficit/hyperactivity disorder (ADD, ADHD), eating disorders (including anorexia nervosa, bulimia nervosa), epilepsy, cognitive disorders (including delirium, substance-induced persisting delirium, dementia, dementia due to HIV disease, dementia due to Huntington's disease, dementia due to Parkinson's disease, dementia of the Alzheimer's type, substance-induced persisting dementia, mild cognitive impairment), personality disorders (including obsessive-compulsive personality disorder, schizoid, schizotypal disorder), substance-related disorders (including alcohol abuse, alcohol dependence, alcohol withdrawal, alcohol withdrawal delirium, alcohol-induced psychotic disorder, amphetamine dependence, amphetamine withdrawal, cocaine dependence, cocaine withdrawal, nicotine dependence, nicotine withdrawal, opioid dependence, opioid withdrawal).

In another specific embodiment, the present invention provides a method for treating schizophrenia or psychosis comprising: administering to a patient in need thereof an effective amount of a compound of the present invention. Particular schizophrenia or psychosis pathologies are paranoid, disorganized, catatonic or undifferentiated schizophrenia and substance-induced psychotic disorder. At present, the text revision of the fourth edition of the Diagnostic and Statistical Manual of Mental Disorders (DSM-IV-TR) (2000, American Psychiatric Association, Washington D.C.) provides a diagnostic tool that includes paranoid, disorganized, catatonic or undifferentiated schizophrenia and substance-induced psychotic disorder. As used herein, the term “schizophrenia or psychosis” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “schizophrenia or psychosis” is intended to include like disorders that are described in other diagnostic sources.

Thus, in an embodiment the present invention provides a method for treating migraine, comprising: administering to a patient in need thereof an effective amount of a compound of the present invention. In one of the available sources of diagnostic tools, Dorland's Medical Dictionary (23'd Ed., 1982, W. B. Saunders Company, Philadelphia, Pa.), migraine is defined as a symptom complex of periodic headaches, usually temporal and unilateral, often with irritability, nausea, vomiting, constipation or diarrhea, and photophobia. As used herein the term “migraine” includes these periodic headaches, both temporal and unilateral, the associated irritability, nausea, vomiting, constipation or diarrhea, photophobia, and other associated symptoms. The skilled artisan will recognize that there are alternative nomenclatures, nosologies, and classification systems for neurological and psychiatric disorders, including migraine, and that these systems evolve with medical scientific progress.

In another specific embodiment, the present invention provides a method for treating anxiety disorders, comprising: administering to a patient in need thereof an effective amount of a compound of the present invention. Particular anxiety disorders are generalized anxiety disorder, obsessive-compulsive disorder and panic attack. At present, the text revision of the fourth edition of the Diagnostic and Statistical Manual of Mental Disorders (DSM-IV-TR) (2000, American Psychiatric Association, Washington D.C.) provides a diagnostic tool that includes anxiety disorders are generalized anxiety disorder, obsessive-compulsive disorder and panic attack. As used herein, the term “anxiety disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “anxiety disorders” is intended to include like disorders that are described in other diagnostic sources.

In another embodiment the present invention provides a method for treating depression, comprising: administering to a patient in need thereof an effective amount of a compound of the present invention. At present, the fourth edition of the Diagnostic and Statistical Manual of Mental Disorders (DSM-IV) (1994, American Psychiatric Association, Washington, D.C.), provides a diagnostic tool including depression and related disorders. Depressive disorders include, for example, single episodic or recurrent major depressive disorders, and dysthymic disorders, depressive neurosis, and neurotic depression; melancholic depression including anorexia, weight loss, insomnia and early morning waking, and psychomotor retardation; atypical depression (or reactive depression) including increased appetite, hypersomnia, psychomotor agitation or irritability, anxiety and phobias; seasonal affective disorder; or bipolar disorders or manic depression, for example, bipolar I disorder, bipolar II disorder and cyclothymic disorder. As used herein the term “depression” includes treatment of those depression disorders and related disorder as described in the DSM-IV.

In another embodiment the present invention provides a method for treating epilepsy, comprising: administering to a patient in need thereof an effective amount of a compound of the present invention. At present, there are several types and subtypes of seizures associated with epilepsy, including idiopathic, symptomatic, and cryptogenic. These epileptic seizures can be focal (partial) or generalized. They can also be simple or complex. Epilepsy is described in the art, such as Epilepsy: A comprehensive textbook. Ed. by Jerome Engel, Jr. and Timothy A. Pedley. (Lippincott-Raven, Philadelphia, 1997). At present, the International Classification of Diseases, Ninth Revision, (ICD-9) provides a diagnostic tool including epilepsy and related disorders. These include: generalized nonconvulsive epilepsy, generalized convulsive epilepsy, petit mal status epilepticus, grand mal status epilepticus, partial epilepsy with impairment of consciousness, partial epilepsy without impairment of consciousness, infantile spasms, epilepsy partialis continua, other forms of epilepsy, epilepsy, unspecified, NOS. As used herein the term “epilepsy” includes these all types and subtypes. The skilled artisan will recognize that there are alternative nomenclatures, nosologies, and classification systems for neurological and psychiatric disorders, including epilepsy, and that these systems evolve with medical scientific progress.

In a specific embodiment, the present invention provides a method for treating cognitive disorders, comprising: administering to a patient in need thereof an effective amount of a compound of the present invention. Particular cognitive disorders are dementia, delirium, amnestic disorders and age-related cognitive decline. At present, the text revision of the fourth edition of the Diagnostic and Statistical Manual of Mental Disorders (DSM-IV-TR) (2000, American Psychiatric Association, Washington D.C.) provides a diagnostic tool that includes cognitive disorders including dementia, delirium, amnestic disorders and age-related cognitive decline. As used herein, the term “cognitive disorders” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “cognitive disorders” is intended to include like disorders that are described in other diagnostic sources.

In another specific embodiment, the present invention provides a method for treating substance-related disorders and addictive behaviors, comprising: administering to a patient in need thereof an effective amount of a compound of the present invention. Particular substance-related disorders and addictive behaviors are persisting dementia, persisting amnestic disorder, psychotic disorder or anxiety disorder induced by substance abuse; and tolerance of, dependence on or withdrawal from substances of abuse. At present, the text revision of the fourth edition of the Diagnostic and Statistical Manual of Mental Disorders (DSM-IV-TR) (2000, American Psychiatric Association, Washington D.C.) provides a diagnostic tool that includes persisting dementia, persisting amnestic disorder, psychotic disorder or anxiety disorder induced by substance abuse; and tolerance of, dependence on or withdrawal from substances of abuse. As used herein, the term “substance-related disorders and addictive behaviors” includes treatment of those mental disorders as described in DSM-IV-TR. The skilled artisan will recognize that there are alternative nomenclatures, nosologies and classification systems for mental disorders, and that these systems evolve with medical and scientific progress. Thus the term “substance-related disorders and addictive behaviors” is intended to include like disorders that are described in other diagnostic sources.

In another specific embodiment, the present invention provides a method for treating pain, comprising: administering to a patient in need thereof an effective amount of a compound of the present invention. Particular pain embodiments are bone and joint pain (osteoarthritis), repetitive motion pain, dental pain, cancer pain, myofascial pain (muscular injury, fibromyalgia), perioperative pain (general surgery, gynecological), chronic pain and neuropathic pain.

The subject compounds are further useful in a method for the prevention, treatment, control, amelioration, or reduction of risk of the diseases, disorders and conditions noted herein.

The subject compounds are further useful in a method for the prevention, treatment, control, amelioration, or reduction of risk of the aforementioned diseases, disorders and conditions in combination with other agents, including an mGluR agonist.

The subject compounds are further useful in a method for the prevention, treatment, control, amelioration, or reduction of risk of the diseases, disorders and conditions noted herein. The subject compounds are further useful in a method for the prevention, treatment, control, amelioration, or reduction of risk of the aforementioned diseases, disorders and conditions in combination with other agents. The compounds of the present invention may be used in combination with one or more other drugs in the treatment, prevention, control, amelioration, or reduction of risk of diseases or conditions for which compounds of the present invention or the other drugs may have utility, where the combination of the drugs together are safer or more effective than either drug alone. Such other drug(s) may be administered, by a route and in an amount commonly used therefor, contemporaneously or sequentially with a compound of the present invention. When a compound of the present invention is used contemporaneously with one or more other drugs, a pharmaceutical composition in unit dosage form containing such other drugs and the compound of the present invention may be desirable. However, the combination therapy may also includes therapies in which the compound of the present invention and one or more other drugs are administered on different overlapping schedules. It is also contemplated that when used in combination with one or more other active ingredients, the compounds of the present invention and the other active ingredients may be used in lower doses than when each is used singly. Accordingly, the pharmaceutical compositions of the present invention include those that contain one or more other active ingredients, in addition to a compound of the present invention. The above combinations include combinations of a compound of the present invention not only with one other active compound, but also with two or more other active compounds. Likewise, compounds of the present invention may be used in combination with other drugs that are used in the prevention, treatment, control, amelioration, or reduction of risk of the diseases or conditions for which compounds of the present invention are useful. Such other drugs may be administered, by a route and in an amount commonly used therefor, contemporaneously or sequentially with a compound of the present invention. Accordingly, the pharmaceutical compositions of the present invention include those that also contain one or more other active ingredients, in addition to a compound of the present invention. The weight ratio of the compound of the present invention to the second active ingredient may be varied and will depend upon the effective dose of each ingredient. Generally, an effective dose of each will be used. Thus, for example, when a compound of the present invention is combined with another agent, the weight ratio of the compound of the present invention to the other agent will generally range from about 1000:1 to about 1:1000, such as about 200:1 to about 1:200. Combinations of a compound of the present invention and other active ingredients will generally also be within the aforementioned range, but in each case, an effective dose of each active ingredient should be used.

In such combinations the compound of the present invention and other active agents may be administered separately or in conjunction. In addition, the administration of one element may be prior to, concurrent to, or subsequent to the administration of other agent(s).

Accordingly, the subject compounds may be used alone or in combination with other agents which are known to be beneficial in the subject indications or other drugs that affect receptors or enzymes that either increase the efficacy, safety, convenience, or reduce unwanted side effects or toxicity of the compounds of the present invention. The subject compound and the other agent may be co-administered, either in concomitant therapy or in a fixed combination.

In one embodiment, the subject compound may be employed in combination with anti-Alzheimer's agents, beta-secretase inhibitors, gamma-secretase inhibitors, HMG-CoA reductase inhibitors, NSAID's including ibuprofen, vitamin E, and anti-amyloid antibodies.

In another embodiment, the subject compound may be employed in combination with sedatives, hypnotics, anxiolytics, antipsychotics, antianxiety agents, cyclopyrrolones, imidazopyridines, pyrazolopyrimidines, minor tranquilizers, melatonin agonists and antagonists, melatonergic agents, benzodiazepines, barbiturates, 5HT-2 antagonists, and the like, such as: adinazolam, allobarbital, alonimid, alprazolam, amisulpride, amitriptyline, amobarbital, amoxapine, aripiprazole, atypical antipsychotics, bentazepam, benzoctamine, brotizolam, bupropion, busprione, butabarbital, butalbital, capuride, carbocloral, chloral betaine, chloral hydrate, clomipramine, clonazepam, cloperidone, clorazepate, chlordiazepoxide, clorethate, chlorpromazine, clozapine, cyprazepam, desipramine, dexclamol, diazepam, dichloralphenazone, divalproex, diphenhydramine, doxepin, estazolam, ethchlorvynol, etomidate, fenobam, flunitrazepam, flupentixol, fluphenazine, flurazepam, fluvoxamine, fluoxetine, fosazepam, glutethimide, halazepam, haloperidol, hydroxyzine, imipramine, lithium, lorazepam, lormetazepam, maprotiline, mecloqualone, melatonin, mephobarbital, meprobamate, methaqualone, midaflur, midazolam, nefazodone, nisobamate, nitrazepam, nortriptyline, olanzapine, oxazepam, paraldehyde, paroxetine, pentobarbital, perlapine, perphenazine, phenelzine, phenobarbital, prazepam, promethazine, propofol, protriptyline, quazepam, quetiapine, reclazepam, risperidone, roletamide, secobarbital, sertraline, suproclone, temazepam, thioridazine, thiothixene, tracazolate, tranylcypromaine, trazodone, triazolam, trepipam, tricetamide, triclofos, trifluoperazine, trimetozine, trimipramine, uldazepam, venlafaxine, zaleplon, ziprasidone, zolazepam, zolpidem, and salts thereof, and combinations thereof, and the like, or the subject compound may be administered in conjunction with the use of physical methods such as with light therapy or electrical stimulation.

In another embodiment, the subject compound may be employed in combination with levodopa (with or without a selective extracerebral decarboxylase inhibitor such as carbidopa or benserazide), anticholinergics such as biperiden (optionally as its hydrochloride or lactate salt) and trihexyphenidyl (benzhexol) hydrochloride, COMT inhibitors such as entacapone, MOA-B inhibitors, antioxidants, A2a adenosine receptor antagonists, cholinergic agonists, NMDA receptor antagonists, serotonin receptor antagonists and dopamine receptor agonists such as alentemol, bromocriptine, fenoldopam, lisuride, naxagolide, pergolide and pramipexole. It will be appreciated that the dopamine agonist may be in the form of a pharmaceutically acceptable salt, for example, alentemol hydrobromide, bromocriptine mesylate, fenoldopam mesylate, naxagolide hydrochloride and pergolide mesylate. Lisuride and pramipexol are commonly used in a non-salt form.

In another embodiment, the subject compound may be employed in combination with a compound from the phenothiazine, thioxanthene, heterocyclic dibenzazepine, butyrophenone, diphenylbutylpiperidine and indolone classes of neuroleptic agent. Suitable examples of phenothiazines include chlorpromazine, mesoridazine, thioridazine, acetophenazine, fluphenazine, perphenazine and trifluoperazine. Suitable examples of thioxanthenes include chlorprothixene and thiothixene. An example of a dibenzazepine is clozapine. An example of a butyrophenone is haloperidol. An example of a diphenylbutylpiperidine is pimozide. An example of an indolone is molindolone. Other neuroleptic agents include loxapine, sulpiride and risperidone. It will be appreciated that the neuroleptic agents when used in combination with the subject compound may be in the form of a pharmaceutically acceptable salt, for example, chlorpromazine hydrochloride, mesoridazine besylate, thioridazine hydrochloride, acetophenazine maleate, fluphenazine hydrochloride, flurphenazine enathate, fluphenazine decanoate, trifluoperazine hydrochloride, thiothixene hydrochloride, haloperidol decanoate, loxapine succinate and molindone hydrochloride. Perphenazine, chlorprothixene, clozapine, haloperidol, pimozide and risperidone are commonly used in a non-salt form. Thus, the subject compound may be employed in combination with acetophenazine, alentemol, aripiprazole, amisulpride, benzhexol, bromocriptine, biperiden, chlorpromazine, chlorprothixene, clozapine, diazepam, fenoldopam, fluphenazine, haloperidol, levodopa, levodopa with benserazide, levodopa with carbidopa, lisuride, loxapine, mesoridazine, molindolone, naxagolide, olanzapine, pergolide, perphenazine, pimozide, pramipexole, quetiapine, risperidone, sulpiride, tetrabenazine, trihexyphenidyl, thioridazine, thiothixene, trifluoperazine or ziprasidone.

In another embodiment, the subject compound may be employed in combination with an anti-depressant or anti-anxiety agent, including norepinephrine reuptake inhibitors (including tertiary amine tricyclics and secondary amine tricyclics), selective serotonin reuptake inhibitors (SSRIs), monoamine oxidase inhibitors (MAOIs), reversible inhibitors of monoamine oxidase (RIMAs), serotonin and noradrenaline reuptake inhibitors (SNRIs), corticotropin releasing factor (CRF) antagonists, α-adrenoreceptor antagonists, neurokinin-1 receptor antagonists, atypical anti-depressants, benzodiazepines, 5-HT_{1A} agonists or antagonists, especially 5-HT_{1A} partial agonists, and corticotropin releasing factor (CRF) antagonists. Specific agents include: amitriptyline, clomipramine, doxepin, imipramine and trimipramine; amoxapine, desipramine, maprotiline, nortriptyline and protriptyline; fluoxetine, fluvoxamine, paroxetine and sertraline; isocarboxazid, phenelzine, tranylcypromine and selegiline; moclobemide: venlafaxine; duloxetine; aprepitant; bupropion, lithium, nefazodone, trazodone and viloxazine; alprazolam, chlordiazepoxide, clonazepam, chlorazepate, diazepam, halazepam, lorazepam, oxazepam and prazepam; buspirone, flesinoxan, gepirone and ipsapirone, and pharmaceutically acceptable salts thereof.

The term “composition” as used herein is intended to encompass a product comprising specified ingredients in predetermined amounts or proportions, as well as any product which results, directly or indirectly, from combination of the specified ingredients in the specified amounts. This term in relation to pharmaceutical compositions is intended to encompass a product comprising one or more active ingredients, and an optional carrier comprising inert ingredients, as well as any product which results, directly or indirectly, from combination, complexation or aggregation of any two or more of the ingredients, or from dissociation of one or more of the ingredients, or from other types of reactions or interactions of one or more of the ingredients. In general, pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

Accordingly, the pharmaceutical compositions of the present invention encompass any composition made by admixing a compound of the present invention and a pharmaceutically acceptable carrier.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

Pharmaceutical compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. Compositions for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Oily suspensions may be formulated by suspending the active ingredient in a suitable oil. Oil-in-water emulsions may also be employed. Dispersible powders and granules suitable for preparation of an aqueous suspension by the addition of water provide the active ingredient in admixture with a dispersing or wetting agent, suspending agent and one or more preservatives.

Pharmaceutical compositions of the present compounds may be in the form of a sterile injectable aqueous or oleagenous suspension. The compounds of the present invention may also be administered in the form of suppositories for rectal administration. For topical use, creams, ointments, jellies, solutions or suspensions, etc., containing the compounds of the present invention may be employed. The compounds of the present invention may also be formulated for administered by inhalation. The compounds of the present invention may also be administered by a transdermal patch by methods known in the art.

The pharmaceutical composition and method of the present invention may further comprise other therapeutically active compounds as noted herein which are usually applied in the treatment of the above mentioned pathological conditions.

The subject compounds are further useful in a method for the prevention, treatment, control, amelioration, or reduction of risk of the diseases, disorders and conditions noted herein. The dosage of active ingredient in the compositions of this invention may be varied, however, it is necessary that the amount of the active ingredient be such that a suitable dosage form is obtained. The active ingredient may be administered to patients (animals and human) in need of such treatment in dosages that will provide optimal pharmaceutical efficacy. The selected dosage depends upon the desired therapeutic effect, on the route of administration, and on the duration of the treatment. The dose will vary from patient to patient depending upon the nature and severity of disease, the patient's weight, special diets then being followed by a patient, concurrent medication, and other factors which those skilled in the art will recognize. Generally, dosage levels of between 0.0001 to 30 mg/kg. of body weight daily are administered to the patient, e.g., humans and elderly humans. The dosage range will generally be about 0.5 mg to 5.0 g. per patient per day which may be administered in single or multiple doses. In one embodiment, the dosage range will be about 0.5 mg to 2.5 mg per patient per day; in another embodiment about 0.5 mg to 1 g per patient per day; in yet another embodiment about 5 mg to 500 mg per patient per day; and in yet another embodiment about 5 mg to 100 mg per patient per day. Pharmaceutical compositions of the present invention may be provided in a solid dosage formulation such as comprising about 0.5 mg to 800 mg active ingredient, or comprising about 1 mg to 400 mg active ingredient. The pharmaceutical composition may be provided in a solid dosage formulation comprising about 1 mg, 5 mg, 10 mg, 25 mg, 50 mg, 100 mg, 200 mg or 250 mg active ingredient. For oral administration, the compositions may be provided in the form of tablets containing 1.0 to 1000 milligrams of the active ingredient, such as 1, 5, 10, 15, 20, 25, 50, 75, 100, 150, 200, 250, 300, 400, 500, 600, 750, 800, 900, and 1000 milligrams of the active ingredient for the symptomatic adjustment of the dosage to the patient to be treated. The compounds may be administered on a regimen of 1 to 4 times per day, such as once or twice per day.

When treating, preventing, controlling, ameliorating, or reducing the risk of neurological and psychiatric disorders associated with glutamate dysfunction or other diseases for which compounds of the present invention are indicated, generally satisfactory results are obtained when the compounds of the present invention are administered at a daily dosage of from about 0.1 milligram to about 100 milligram per kilogram of animal body weight, preferably given as a single daily dose or in divided doses two to six times a day, or in sustained release form. For most large mammals, the total daily dosage is from about 1.0 milligrams to about 5000 milligrams, preferably from about 1 milligrams to about 1000 milligrams. In the case of a 70 kg adult human, the total daily dose will generally be from about 7 milligrams to about 800 milligrams. This dosage regimen may be adjusted to provide the optimal therapeutic response.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

Several methods for preparing the compounds of this invention are illustrated in the following Schemes and Examples. Starting materials and the requisite intermediates are in some cases commercially available, or can be prepared according to literature procedures or as illustrated herein. The compounds of this invention may be prepared by employing reactions as shown in the following schemes, in addition to other standard manipulations that are known in the literature or exemplified in the experimental procedures. Substituent numbering as shown in the schemes does not necessarily correlate to that used in the claims and often, for clarity, a single substituent is shown attached to the compound where multiple substituents are allowed under the definitions hereinabove. Reactions used to generate the compounds of this invention are prepared by employing reactions as shown in the schemes and examples herein, in addition to other standard manipulations such as ester hydrolysis, cleavage of protecting groups, etc., as may be known in the literature or exemplified in the experimental procedures. Starting materials are made according to procedures known in the art or as illustrated herein. The following abbreviations are used herein: Me: methyl; Et: ethyl; t-Bu: tert-butyl; Ar: aryl; Ph: phenyl; Bn: benzyl; Ac: acetyl; THF: tetrahydrofuran; DIEA: N,N-diisopropylethylamine; DMSO: dimethylsulfoxide; EDC: N-(3-dimethylaminopropyl)-N′-ethylcarbodiimide hydrochloride; HOBT: hydroxybenzotriazole hydrate; Boc: tert-butyloxy carbonyl; Et₃N: triethylamine; EtOAc: ethyl acetate; CH₂Cl₂: dichloromethane; CH₃OH: methanol; C₂H₅OH: ethanol; CH₃CN: acetonitrile; BSA: bovine serum albumin; TFA: trifluoracetic acid; DMF: N,N-dimethylformamide; MTBE: methyl tert-butyl ether; SOCl₂: thionyl chloride; CDI: carbonyl diimidazole; RT: room temperature; HPLC: high performance liquid chromatography; TEMPO: 2,2,6,6,-tetramethyl-1-piperidine 1-oxyl; HATU: O-(7-azabenzotriazol-1-yl)-N,N,′,′-tetramethyluronium hexafluorophosphate; Burgess reagent: methoxycarbonylsulfamoyl) trimethylammonium inner salt. The compounds of the present invention can be prepared in a variety of fashions.

In some cases the final product may be further modified, for example, by manipulation of substituents. These manipulations may include, but are not limited to, reduction, oxidation, alkylation, acylation, and hydrolysis reactions which are commonly known to those skilled in the art. In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

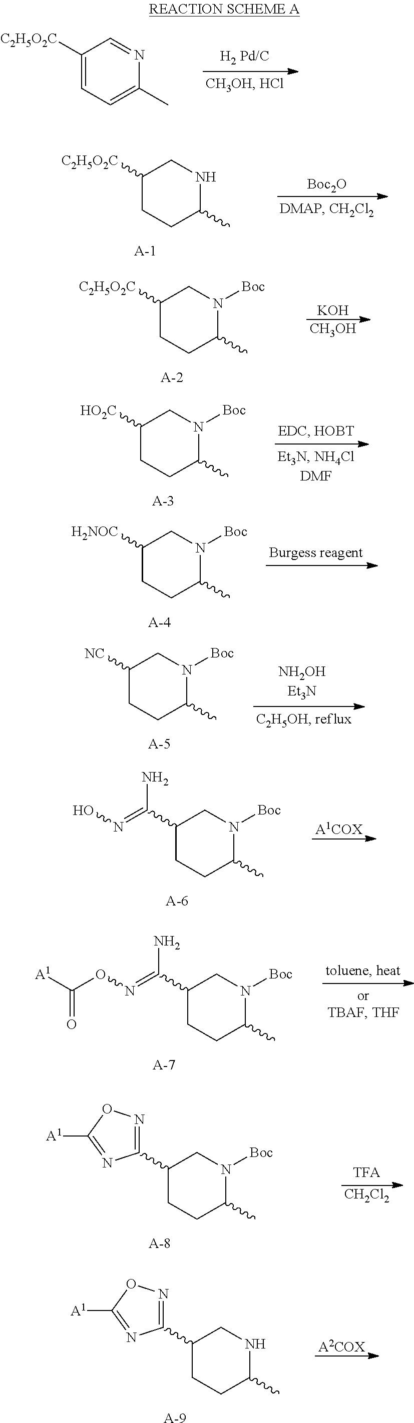

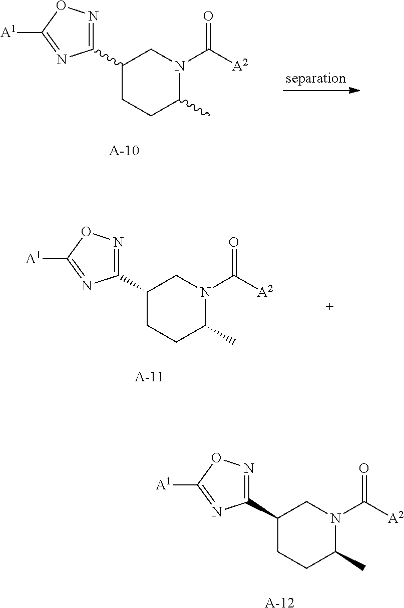

As shown in Reaction Scheme A, esters of nicotinic acid substituted on the pyridine ring can undergo reduction to provide substituted piperidine-3-carboxylate esters such as A-1, as a mixture of cis/trans isomers. Following protection of the piperidine nitrogen, the ester is converted to the primary amide A-4 using standard methodology such as hydrolysis to carboxylic acid A-3 and coupling to ammonia. Dehydration of primary amides to nitriles is accomplished under a variety of well-known conditions. The dehydration of A-4 to nitrile A-5 proceeds with Burgess reagent, according to methodology described in Claremon and Phillips, Tetrahedron Lett, 1988, 29(18), 2155-2158. Reaction with hydroxylamine yields hydroxyamidine A-6, which is acylated under standard conditions such as reaction with an acid chloride, or by reaction with a carboxylic acid and amide coupling reagents, to give acyl hydroxyamidine A-7. Heating a solution of A-7 effects cyclization to oxadiazole A-8. The same transformation is also be accomplished using TBAF in a solvent such as THF, according to methodology reported in Gangloff et al, Tetrahedron Lett. 2001, 42, 1441-1443. In the case of the Boc-protected A-8, deprotection of the piperidine nitrogen is accomplished with acid to yield the free amine A-9. The latter is acylated using standard acylating agents and conditions, such as reaction with an acid chloride in the presence of a base to give carboxamide A-10. Alternatively, A-9 is coupled to a carboxylic acid using amide coupling reagents such as EDC with HOBT, to give amide A-10. In Scheme A, separation of the cis/trans isomers that are generated during reduction of the pyridine in the first step generally is accomplished by silica gel column chromatography or reverse phase HPLC of a compound in the sequence from A-1 through A-10. The racemic cis product is further separated by chiral chromatography to give the individual cis enantiomers A-11 and A-12. Other methods to effect chiral resolution may also be employed, such as crystallization of a diastereomeric salt of an appropriate synthetic intermediate or product.

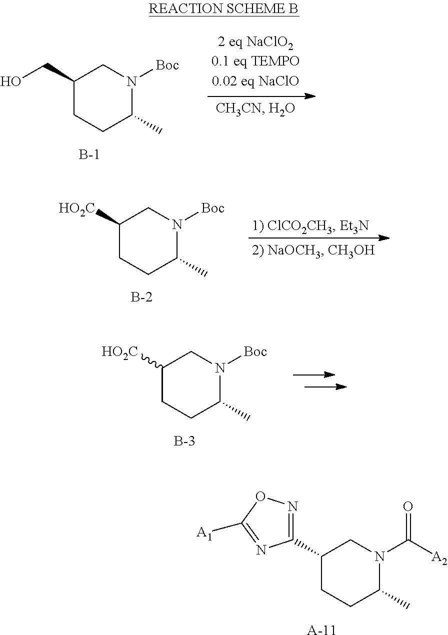

As shown in Reaction Scheme B, the chiral hydroxymethylpiperidine intermediate B-1 (prepared as described in PCT Publication WO 2008/147518, Dec. 4, 2008) is oxidized to the corresponding carboxylic acid B-2 and subsequently epimerized to B-3 upon sequential treatment with methylchloroformate and a base, followed by sodium methoxide in methanol, first at RT and then reflux. B-3 is converted to the final desired products using the chemistry outlined in Scheme A.

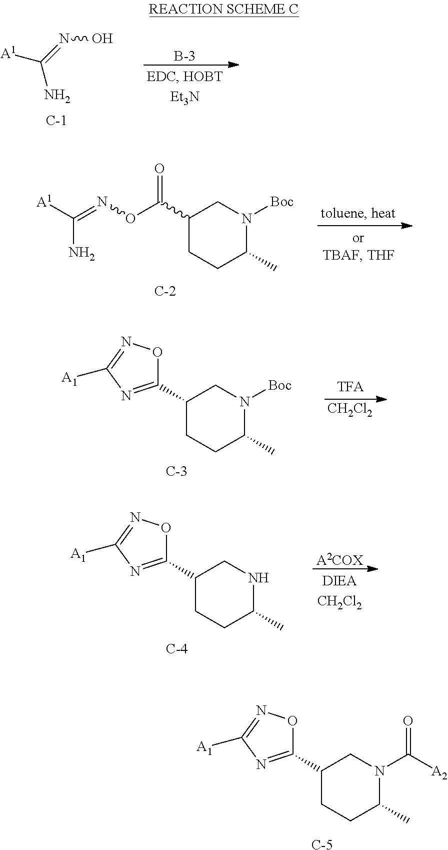

As shown in Reaction Scheme C, hydroxyamidines C-1 are acylated by piperidine carboxylic acid B-3 using amide coupling procedures such as EDC with HOBT, to give C-2. Cyclization of C-2 is accomplished as described previously for Reaction Scheme A, using either thermal conditions or TBAF catalyst. Conversion to the final products C-5 is accomplished using chemistry described in Reaction Scheme A.

### EXAMPLE 1

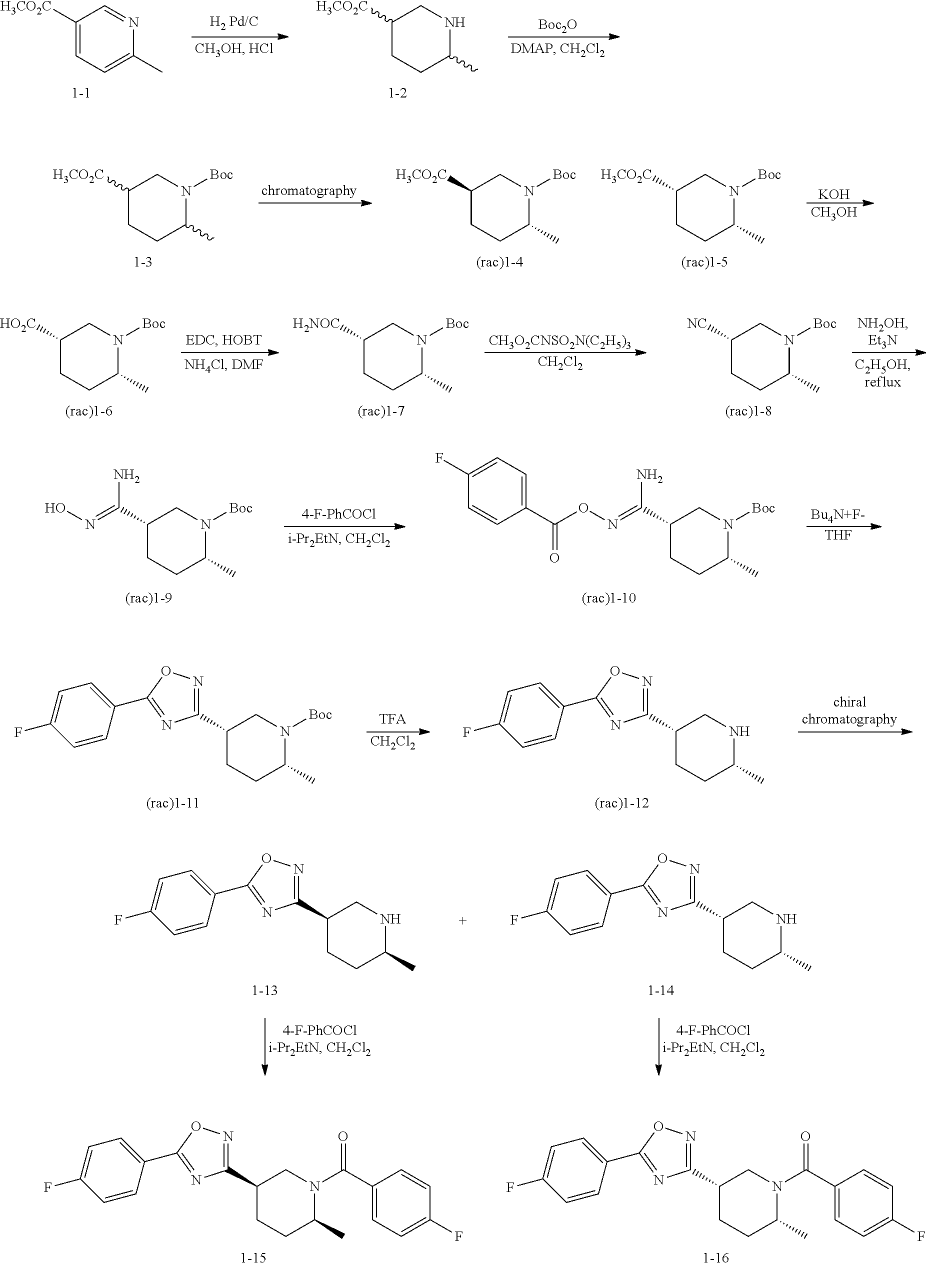

### Methyl 6-methylpiperidine-3-carboxylate (1-2)

Methyl 6-methylnicotinate 1-1 (6 g, 39.7 mmol) was dissolved in CH₃OH (100 mL), and conc. HCl solution (4 mL) was added. The resulting solution was de-oxygenated prior to the addition of 10% palladium on carbon (2.112 g, 1.985 mmol), and the reaction mixture was stirred under 49 psi hydrogen for 24 h. Hydrogen was purged from the reaction mixture, and the catalyst was removed by filtration. The organic solution was concentrated and 1-2 was used directly in the next step.

### 1-tert-Butyl trans-3-methyl 6-methylpiperidine-1,3-dicarboxylate (1-4) and 1-tert-butyl cis-3-methyl 6-methylpiperidine-1,3-dicarboxylate (1-5)

Di-tert-butyl dicarbonate (2.80 g, 12.85 mmol) and 4-dimethylaminopyridine (0.157 g, 1.285 mmol) was added to a solution of 1-2 (2.02 g, 12.85 mmol) in CH₂Cl₂ (50 mL). The resulting mixture was stirred over night. The reaction mixture was washed with Na₂HCO₃ solution, brine; dried over Na₂SO₄, filtered and concentrated to give product 1-3 as a mixture of cis/trans products. The trans and cis isomers were separated by reverse phase HPLC (C-18, 100×30 mm column, gradient elution with 5% to 95% CH₃CN in H₂O (0.1% TFA)) to provide trans-1-tert-butyl 3-methyl 6-methylpiperidine-1,3-dicarboxylate 1-4, and cis-1-tert-butyl 3-methyl 6-methylpiperidine-1,3-dicarboxylate 1-5.

### cis-1-(tert-Butoxycarbonyl)-6-methylpiperidine-3-carboxylic acid (1-6)

Potassium hydroxide (331 mg, 5.90 mmol) was added to a solution of 1-5 (800 mg, 2.95 mmol) in CH₃OH (20 mL) and the resulting mixture stirred at 60° C. for 3 h. The reaction was cooled to −45° C. and conc. HCl (0.491 ml, 5.90 mmol) in methanol (10 mL) was added dropwise. The resulting mixture was concentrated to give 1-6 which was used directly in the next step.

### tert-Butyl cis-5-(aminocarbonyl)-2-methylpiperidine-1-carboxylate (1-7)

DIEA (1.120 mL, 6.43 mmol), HOBT (542 mg, 3.54 mmol) and EDC (678 mg, 3.54 mmol) were added to a solution of 1-6 (717 mg, 2.95 mmol) in DMF (8 mL). The resulting solution was stirred for 10 min before adding ammonium chloride (315 mg, 5.89 mmol). The resulting mixture was stirred over night at RT. Water was added and the mixture extracted with EtOAc. The organic phase was washed with brine, dried over Na₂SO₄ and concentrated. The crude product was purified by column chromatography on silica gel (80 g column, eluting with 0 to 5% CH₃OH in CH₂Cl₂) to give 1-7.

### tert-Butyl cis-5-cyano-2-methylpiperidine-1-carboxylate (1-8)

Burgess reagent (2.107 g, 8.84 mmol) was added in 3 equal portions over the course of 1 h to a solution of 1-7 (714 mg, 2.95 mmol) in CH₂Cl₂ (50 mL) under nitrogen. The reaction was stirred 2 h and then diluted with CH₂Cl₂ (50 mL) and washed with brine (2×25 mL) and dried over Na₂SO₄. The crude product was purified by silica gel column chromatography (80 g column, eluting with 0 to 30% EtOAc in hexane) to give 1-8.

### tert-Butyl cis-5-[amino(hydroxyimino)methyl]-2-methylpiperidine-1-carboxylate (1-9)

Hydroxylamine hydrochloride (256 mg, 3.68 mmol) was added to a stirred solution of 1-8 (550 mg, 2.452 mmol) and Et₃N (0.502 ml, 3.68 mmol) in C₂H₅OH (40 mL). The reaction mixture was refluxed over night. The solvent was evaporated in vacuo and the crude product was purified by silica gel chromatography (80 g column, eluting with 0 to 5% CH₃OH in CH₂Cl₂) to provide 1-9.

### tert-Butyl cis-5-(amino{[(4-fluorobenzoyl)oxy]imino}methyl)-2-methylpiperidine-1-carboxylate (1-10)

A stirred solution of 1-9 (480 mg, 1.865 mmol) in CH₂Cl₂ (40 mL) was cooled to −0° C. 4-Fluorobenzoyl chloride (0.224 ml, 1.865 mmol) and DIEA (0.325 ml, 1.865 mmol) were added to the reaction. The reaction was allowed to warm to RT and stirred for 1 h, then diluted with CH₂Cl₂ (10 mL), washed with brine, dried over Na₂SO₄ and concentrated. The crude product was chromatographed on silica gel (40 g column, eluting with 0 to 5% CH₃OH in CH₂Cl₂) to give 1-10.

### tert-Butyl cis-5-[5-(4-fluorophenyl)-1,2,4-oxadiazol-3-yl]-2-methylpiperidine-1-carboxylate (1-11)

TBAF (798 mg, 2.53 mmol) was added to a stirred solution of 1-11 (480 mg, 1.265 mmol) in THF (20 mL) and the reaction mixture heated at 40° C. for 2 h. The THF was evaporated and the crude product was chromatographed on silica gel (40 g column, eluting with 0 to 5% CH₃OH in CH₂Cl₂) to give 1-11.

### (2S,5R)-5-[5-(4-Fluorophenyl)-1,2,4-oxadiazol-3-yl]-2-methylpiperidine (1-13) and (2R,5S)-5-[5-(4-fluorophenyl)-1,2,4-oxadiazol-3-yl]-2-methylpiperidine (1-14)

A solution of 1-11 (380 mg, 1.051 mmol) in 1:1 TFA/CH₂Cl₂ (4 mL), was stirred at RT for 30 min. The TFA/CH₂Cl₂ was removed in vacuo and the crude racemic product 1-12 was separated by chiral column chromatography to give 1-13 and 1-14.

### (2S,5R)-1-(4-fluorobenzoyl)-5-[5-(4-fluorophenyl)-1,2,4-oxadiazol-3-yl]-2-methylpiperidine (1-15)

4-Fluorobenzoyl chloride (0.034 mL, 0.287 mmol) and DIEA (0.050 mL, 0.287 mmol) were added to a stirred solution of 1-13 (50 mg, 0.191 mmol) in CH₂Cl₂ at −15° C., and the reaction mixture allowed to warm to RT. The crude product was loaded directly on a silica gel column and chromatographed (12 g column, eluting with 0 to 5% CH₃OH in CH₂Cl₂). The fractions containing the desired product were concentrated and purified by reverse phase HPLC to provide 1-15.

### (2R,5S)-1-(4-fluorobenzoyl)-5-[5-(4-fluorophenyl)-1,2,4-oxadiazol-3-yl]-2-methylpiperidine (1-16)

4-Fluorobenzoyl chloride (0.034 ml, 0.287 mmol) and DIEA (0.050 ml, 0.287 mmol) were added to a stirred solution of 1-14 (50 mg, 0.191 mmol) in CH₂Cl₂ (4 mL) at −15° C., and the reaction mixture allowed to warm to RT. The crude product was loaded directly on a silica gel column and chromatographed (12 g column, eluting with 0 to 5% CH₃OH in CH₂Cl₂). The fractions containing the desired product were concentrated and purified by reverse phase HPLC to provide 1-16. MS m/z (M+H) 384.1445 found, 383.1445 required.

### EXAMPLE 2

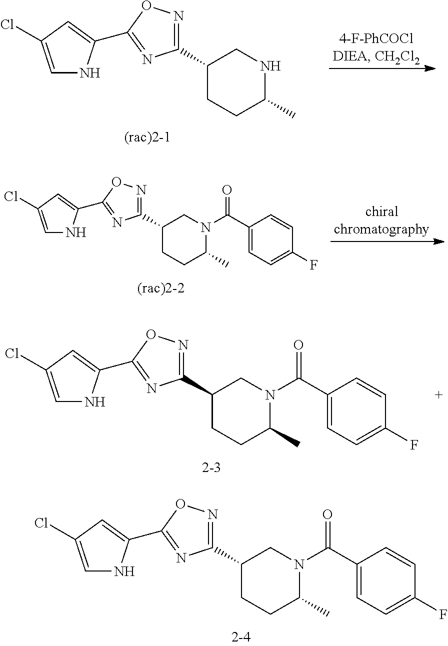

### (2S,5R)-5-[5-(4-chloro-1H-pyrrol-2-yl)-1,2,4-oxadiazol-3-yl]-1-(4-fluorobenzoyl)-2-methylpiperidine (2-3) and (2R,5S)-5-[5-(4-chloro-1H-pyrrol-2-yl)-1,2,4-oxadiazol-3-yl]-1-(4-fluorobenzoyl)-2-methylpiperidine (2-4)

(2R,5S)-5-[5-(4-Chloro-1H-pyrrol-2-yl)-1,2,4-oxadiazol-3-yl]-2-methylpiperidine (2-1) was prepared according to Example 1, using 4-chloropyrrolecarboxylic acid (preparation: WO 2006/123257, Nov. 23, 2006) in place of 4-fluorobenzoic acid. Compound 2-1 (50 mg, 0.187 mmol) was dissolved in CH₂Cl₂ (10 mL). The resulting stirred solution was cooled to −15° C., and 4-fluorobenzoyl chloride (0.027 ml, 0.225 mmol) and DIEA (0.039 ml, 0.225 mmol) were added. The reaction mixture was allowed to warm to RT and stirred for 1 h. The crude product was purified by silica gel chromatography (12 g column, 0 to 5% CH₃OH in CH₂Cl₂), followed by reverse phase HPLC. The enantiomers were separated by chiral chromatography, giving 2-3 and 2-4. 2-3: MS m/z (M+H) 389.1186 found, 389.1102 required. 2-4: MS m/z (M+H) 389.1182 found, 389.1102 required.

### EXAMPLE 3

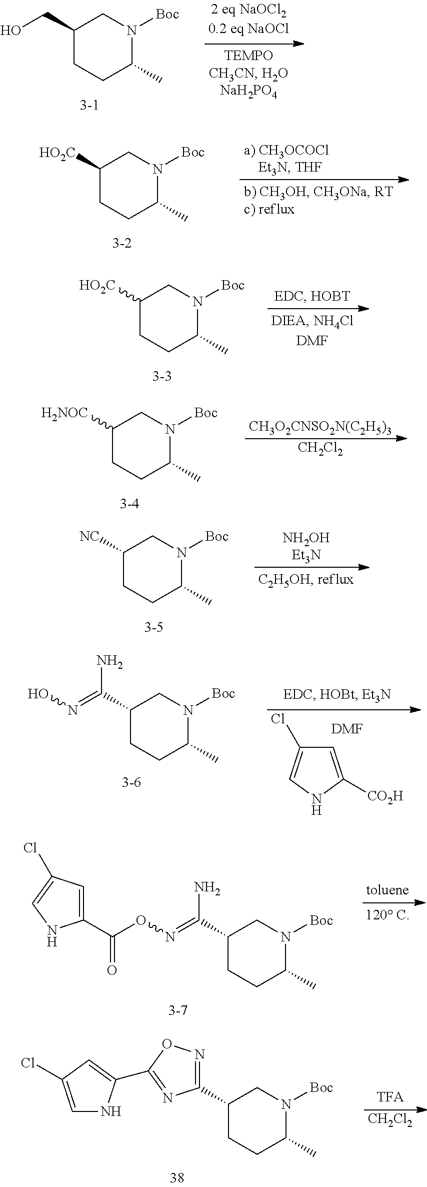

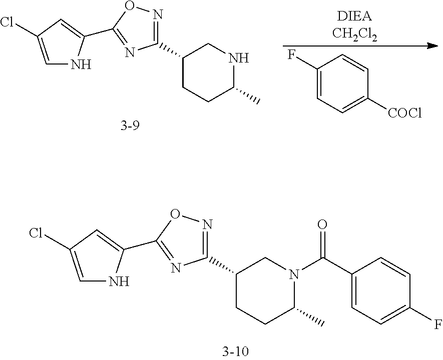

### (3R,6R)-1-(tert-butoxycarbonyl)-6-methylpiperidine-3-carboxylic acid (3-2)

tert-Butyl (2R,5R)-5-(hydroxymethyl)-2-methylpiperidine-1-carboxylate (3-1) (15.29 g, 66.7 mmol), TEMPO (1.04 g, 6.67 mmol), CH₃CN (350 ml) and sodium phosphate buffer (250 ml, pH=6.7) is heated to 35° C. A solution of sodium chlorite (12.06 g, 133 mmol) in water (70%) and sodium hypochlorite (0.752 mL, 1.334 mmol) in water (35 mL) were added simultaneously over 2 hours. The mixture was stirred at 35° C. until the reaction was complete, then cooled to RT. Most of the CH₃CN was removed in vacuo, and the remainder was extracted with EtOAc. The organic layer was washed with saturated Na₂SO₃ and brine, dried over Na₂SO₄ and concentrated to give 3-2.

### (3S,6R)-1-(tert-Butoxycarbonyl)-6-methylpiperidine-3-carboxylic acid (3-3)

Intermediate 3-2 was dissolved in THF (500 mL), triethylamine (10.70 ml, 77 mmol) was added and the resulting mixture was cooled to −4° C. Methyl chloroformate (5.91 mL, 77 mmol) was added. The reaction mixture was allowed to warm to RT, and a white solid formed. The solid was filtered and the mother liquid was concentrated. This was dissolved in CH₃OH (500 mL) under nitrogen. Sodium methoxide (52.7 g, 230 mmol) was added and the resulting solution was stirred for 72 h. The reaction mixture was heated to reflux for 6 h and then concentrated. The mixture was suspended in CH₃OH (20 mL) and refluxed for another 6 h then concentrated. The crude product was dissolved in water (20 mL), ice was added followed by 1N HCl. The solid that formed was collected by filtration and dried to give a 4:1 mixture of 3-3 and 3-2.

### tert-Butyl 5-(aminocarbonyl)-2-methylpiperidine-1-carboxylate (3-4)

DIEA (15.75 mL, 90 mmol), HOBT (7.62 g, 49.7 mmol) and EDC (9.53 g, 49.7 mmol) were added to a solution of 4:1 3-3 and 3-2 (11 g, 45.2 mmol) in DMF (50 mL). The resulting solution was stirred for 10 min before adding ammonium chloride (7.26 g, 136 mmol). The resulting mixture was stirred over night at RT. Brine (150 mL) was added and the mixture extracted with EtOAc (2×40 mL). The organic phase was washed with brine, dried over Na₂SO₄ and concentrated. The crude product, a mixture of tert-butyl (2R,5S)-5-(aminocarbonyl)-2-methylpiperidine-1-carboxylate and tert-butyl (2R,5R)-5-(aminocarbonyl)-2-methylpiperidine-1-carboxylate, was used in the next step.

### tert-Butyl (2R,5S)-5-cyano-2-methylpiperidine-1-carboxylate (3-5)

Burgess reagent (25.4 g, 106 mmol) was added in 5 equal portions over the course of 1 h to a solution of 3-4 and tert-butyl (2R,5R)-5-(aminocarbonyl)-2-methylpiperidine-1-carboxylate (12.89 g, 53.2 mmol) in CH₂Cl₂ (500 mL) under nitrogen. The reaction was stirred overnight and then diluted with CH₂Cl₂ (50 mL) and washed with brine (2×25 mL) and dried over Na₂SO₄. The crude product was purified by silica gel column chromatography (330 g column, eluting with 0 to 30% EtOAc in hexane) to give 3-5.

### tert-Butyl (2R,5S)-5-[(Z,E)-amino(hydroxyimino)methyl]-2-methylpiperidine-1-carboxylate (3-6)

Hydroxylamine hydrochloride (1.929 g, 27.8 mmol) was added to a stirred solution of 3-5 (5.66 g, 25.2 mmol) and Et₃N (3.79 ml, 27.8 mmol) in C₂H₅OH (160 mL). The reaction mixture was sealed and stirred at 80° C. for 4 h. The solvent was evaporated in vacuo and the crude product was dissolved in H₂O/C₂H₅OH (90/10, 100 mL) and left to stand overnight. The resulting crystals were filtered to give 3-6.

### tert-Butyl (2R,5S)-5-[(Z)-amino({[(4-chloro-1H-pyrrol-2-yl)carbonyl]oxy}imino)methyl]-2-methylpiperidine-1-carboxylate (3-7)

4-Chloropyrrole-2-carboxylic acid (1.244 g, 8.55 mmol) was dissolved in DMF (15 mL). To this solution was added EDC (1.788 g, 9.33 mmol), HOBT (1.428 g, 9.33 mmol) and triethylamine (1.272 mL, 9.33 mmol) The resulting solution was stirred for 10 min and 3-6 (2.0 g, 7.77 mmol) was added. The resulting mixture was stirred over night at RT and diluted with water. It was extracted with EtOAc and the organic phase was combined and washed with brine, dried over Na₂SO₄ and concentrated. The crude product was purified by silica gel column chromatography (120 g column, 0 to 10% CH₃OH in CH₂Cl₂) to give 3-7.

### tert-Butyl (2R,5S)-5-[5-(4-chloro-1H-pyrrol-2-yl)-1,2,4-oxadiazol-3-yl]-2-methylpiperidine-1-carboxylate (3-8)

Intermediate 3-7 (2.9 g, 7.54 mmol) was dissolved in toluene (5 mL). The reaction mixture was stirred at 120° C. overnight. The solvent was removed in vacuo to give 3-8.

### (2R,5S)-5-[5-(4-chloro-1H-pyrrol-2-yl)-1,2,4-oxadiazol-3-yl]-2-methylpiperidine (3-9)

Intermediate 3-8 (2.5 g, 6.81 mmol) was dissolved in CH₂Cl₂ (10 mL), and TFA (5 mL) was added. The resulting mixture was stirred for 30 min. The reaction was concentrated in vacuo and the crude product was dissolved in CH₂Cl₂ and washed with aq. NaHCO₃ and brine, and dried over Na₂SO₄ and concentrated. The crude product was purified by silica gel column chromatography (40 g column, 0 to 10% CH₃OH in CH₂Cl₂) to give 3-9.

### (2R,5S)-5-[5-(4-chloro-1H-pyrrol-2-yl)-1,2,4-oxadiazol-3-yl]-1-(4-fluorobenzoyl)-2-methylpiperidine (3-10)

Intermediate 3-9 (330 mg, 1.237 mmol) was dissolved in CH₂Cl₂ (20 mL). The resulting solution was cooled to −15° C., and 4-fluorobenzoyl chloride (0.178 ml, 1.485 mmol) and DIEA (0.259 mL, 1.485 mmol) were added. The reaction mixture was allowed to warm to RT and stirred for 1 h. The reaction was concentrated and the crude product was purified by silica gel column chromatography (40 g column, 0 to 5% CH₃OH in CH₂Cl₂) and further purified by reverse phase HPLC (C-18, 100×30 mm column, gradient elution with 5% to 95% CH₃CN in H₂O (0.1% TFA)) to give 3-10 (2-4). MS m/z (M+H) 389.116 found, 389.1175 required.

### EXAMPLE 4

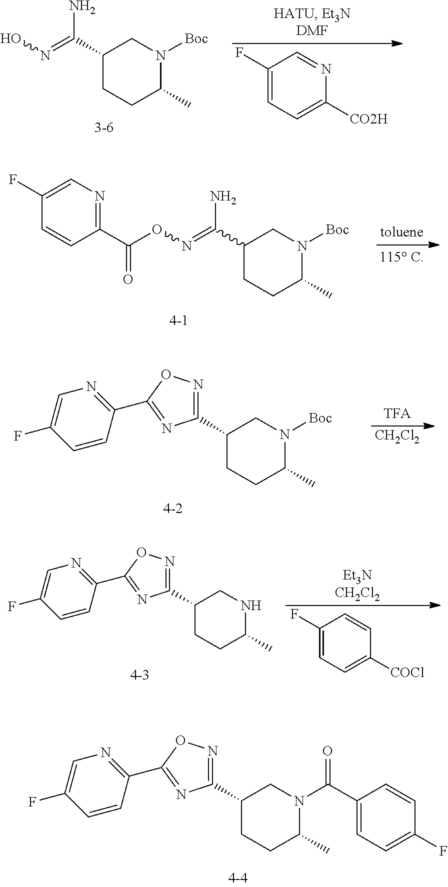

### tert-Butyl (2R,5S)-5-[(Z,E)-amino({[(5-fluoropyridin-2-yl)carbonyl]oxy}imino)methyl]-2-methylpiperidine-1-carboxylate (4-1)

Intermediate 3-6 (274 mg, 1.063 mmol) and 5-fluoropyridine-2-carboxylic acid (150 mg, 1.063 mmol) were combined in DMF (10 mLl), and to this was added Et₃N (0.319 mL, 2.339 mmol) and HATU (485 mg, 1.276 mmol). The resulting mixture was stirred for 5 min. The reaction mixture was poured into water (100 mL) and extracted with EtOAc (2×20 mL). The organic phase was combined and washed with brine, dried over Na₂SO₄ and concentrated. The crude product was dissolved in CH₃CN and left standing at RT. The crystals that formed were collected by filtration and washed with water and dried to give 4-1 which was used directly in the next step.

### tert-Butyl (2R,5S)-5-[5-(5-fluoropyridin-2-yl)-1,2,4-oxadiazol-3-yl]-2-methylpiperidine-1-carboxylate (4-2)

Intermediate 4-1 (400 mg, 1.051 mmol) was added to toluene (60 mL), along with 3 Å molecular sieves (2 g). The resulting mixture was stirred for 6 h at 115° C. The reaction mixture was filtered and the filter cake was washed with EtOAc. Concentration of the filtrate gave the crude product which was purified by silica gel column chromatography (40 g column, 0 to 5% CH₃OH in CH₂Cl₂) to give 4-2.

### 5-Fluoro-2-{3-[(3S,6R)-6-methylpiperidin-3-yl]-1,2,4-oxadiazol-5-yl}pyridine (4-3)

Intermediate 4-2 (298 mg, 0.822 mmol) was dissolved in CH₂Cl₂ (5 mL), and TFA (1 mL) in CH₂Cl₂ (2 mL) was added. The resulting mixture was allowed to stand overnight at RT. The reaction was concentrated to give the crude product 4-3.

### 5-Fluoro-2-{3-[(3S,6R)-1-(4-fluorobenzoyl)-6-methylpiperidin-3-yl]-1,2,4-oxadiazol-5-yl}pyridine (4-4)

Intermediate 4-3 (370 mg, 0.811 mmol) and Et₃N (0.332 mL, 2.433 mmol) were combined with CH₂Cl₂ (20 mLl), and cooled to 0° C. 4-Fluorobenzoyl chloride (0.117 mL, 0.973 mmol) in CH₂Cl₂ (2 mL) was added to above solution and allowed to warm to RT, then stirred for 30 min. The reaction was concentrated and the crude product was dissolved in CH₃CN/water and purified by reverse phase HPLC (C-18, 100×30 mm column, gradient elution with 5% to 95% CH₃CN in H₂O (0.1% TFA)) to give 4-4. MS m/z (M+H) 385.1475 found, 385.1398 required.

### EXAMPLE 5

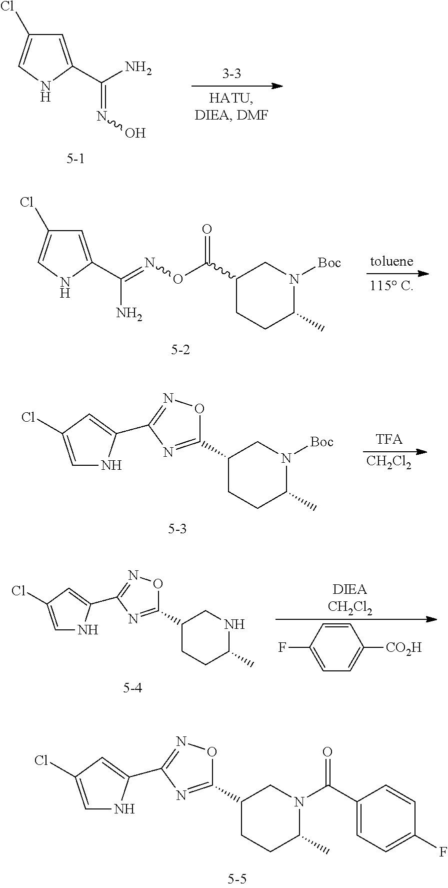

### tert-Butyl (2R,5RS)-5-[({[(1-Z,E)-amino(4-chloro-1H-pyrrol-2-yl)methylene]amino}oxy)carbonyl]-2-methylpiperidine-1-carboxylate (5-2)

Intermediate 3-3 (3.21 g, 13.19 mmol) was dissolved in DMF (20 mL) and to this was added DIEA (4.60 mL, 26.4 mmol), 4-chloro-N′-hydroxy-1H-pyrrole-2-carboximidamide 5-1 (2.74 g, 17.15 mmol) (preparation: WO 2006/123257, Nov. 23, 2006) and HATU (6.52 g, 17.15 mmol). The resulting mixture was stirred for 30 min. The reaction was poured into brine (200 mL) and extracted with EtOAc. The EtOAc layer was washed with brine. The crude was purified by silica gel column chromatography (330 g column, 0 to 5% CH₃OH in CH₂Cl₂) to give 5-2.

### tert-Butyl (2R,5S)-5-[3-(4-chloro-1H-pyrrol-2-yl)-1,2,4-oxadiazol-5-yl]-2-methylpiperidine-1-carboxylate (5-3)

Intermediate 5-2 (3.8 g, 9.87 mmol) was dissolved in toluene (100 mL) and 3 Å molecular sieves (0.1 g) was added. This mixture was stirred at 115° C. for 3 h. The reaction was filtered and the filtrate was concentrated. The crude product was purified by silica gel column chromatography (120 g column, 0 to 30% EtOAc in hexane) and pure cis isomer 5-3 was isolated.

### (2R,5S)-5-[3-(4-Chloro-1H-pyrrol-2-yl)-1,2,4-oxadiazol-5-yl]-2-methylpiperidine (5-4)

Intermediate 5-3 (2.6 g, 7.09 mmol) was dissolved in CH₂Cl₂ (20 mL) and cooled to 0° C. A 1:1 solution of CH₂Cl₂ and TFA (10 mL) was added and the reaction allowed to warm to RT. After stirring 30 min. saturated sodium bicarbonate was added to neutralize the reaction, and the resulting mixture was washed with brine. The organic solution was dried over sodium sulfate and concentrated to give 5-4.

### (2R,5S)-5-[3-(4-Chloro-1H-pyrrol-2-yl)-1,2,4-oxadiazol-5-yl]-1-(4-fluorobenzoyl)-2-methylpiperidine (5-5)

Intermediate 5-4 (300 mg, 1.125 mmol) was dissolved in CH₂Cl₂ (30 mL) and cooled to −0° C. To this solution was added DIEA (0.235 mLl, 1.350 mmol) and 4-fluorobenzoyl chloride (0.162 mL, 1.350 mmol) in dry CH₂Cl₂ (2 mL). The reaction was allowed to warm to RT and stirred for 1 h. The reaction was concentrated and the crude product was dissolved in CH₂Cl₂ and chromatographed on silica gel (40 g column, 0 to 5% CH₃OH in CH₂Cl₂) followed by reverse phase HPLC (C-18, 100×30 mm column, gradient elution with 5% to 95% CH₃CN in H₂O (0.1% TFA)) to give 5-5. MS m/z (M+H) 389.1178 found, 389.1102 required.

### EXAMPLE 6

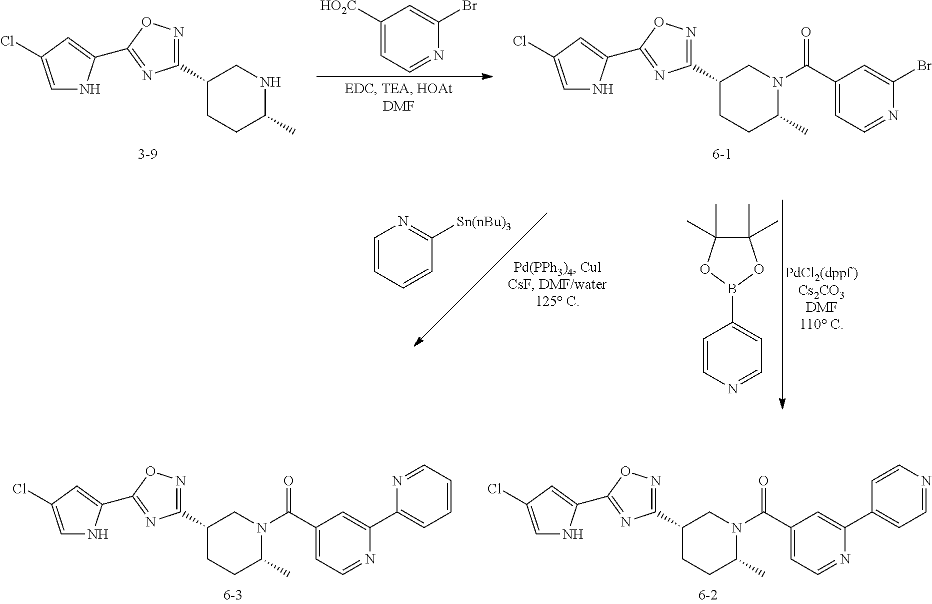

### (2-bromopyridin-4-yl){(2R,5S)-5-[5-(4-chloro-1H-pyrrol-2-yl)-1,2,4-oxadiazol-3-yl]-2-methylpiperidin-1-yl}methanone (6-1)

To a solution of intermediate 3-9 (250 mg, 0.937 mmol) in DMF (3.7 mL) was added 2-bromopyridine-4-carboxylic acid (284 mg, 1.41 mmol), EDC (359 mg, 1.88 mmol), HOAt (255 mg, 1.88 mmol), and TEA (523 μL, 3.75 mmol) and the reaction was stirred at ambient temperature for 2 h. The reaction was diluted with EtOAc (30 mL), and the organic phase was washed with saturated NaHCO3 (2×30 mL), water (2×30 mL), and brine (2×30 mL). The combined organics were dried over magnesium sulfate, filtered, and concentrated. The crude mixture was purified via normal phase column chromatography (silica, 5% to 85% EtOAc in hexanes) to afford 6-1 as a white solid. MS m/z (M+H) 450.0322 found, 450.0327 required.

### 2,4′-bipyridin-4-yl{(2R,5S)-5-[5-(4-chloro-1H-pyrrol-2-yl)-1,2,4-oxadiazol-3-yl]-2-methylpiperidin-1-yl}methanone (6-2)

To a solution of intermediate 6-1 (20 mg, 0.044 mmol) in DMF/water (4:1, 0.44 mL) was added PdCl₂(dppf) (9.8 mg, 0.013 mmol), cesium carbonate (36 mg, 0.11 mmol) and pyridine-4-boronic acid pinacol ester (18 mg, 0.089 mmol) and the reaction was heated to 110° C. in a microwave reactor for 15 minutes. The reaction was cooled and partitioned between EtOAc (10 mL) and water (10 mL). The organic phase was washed with water (2×10 mL) and brine (10 mL). The combined organics were dried over magnesium sulfate, filtered, and concentrated. The crude mixture was purified via normal phase column chromatography (silica, 0% to 15% MeOH in EtOAc) to afford 6-2 as a foamy solid. MS m/z (M+H) 449.1478 found, 449.1487 required.

### 2,2′-bipyridin-4-yl{(2R,5S)-5-[5-(4-chloro-1H-pyrrol-2-yl)-1,2,4-oxadiazol-3-yl]-2-methylpiperidin-1-yl}methanone (6-3)

To a solution of intermediate 6-1 (25 mg, 0.055 mmol) in DMF (0.56 mL) was added Pd(PPh₃)₄ (6.4 mg, 0.0055 mmol), copper (I) iodide (2.1 mg, 0.011 mmol), cesium fluoride (25 mg, 0.17 mmol), and 2-(tributylstannyl)pyridine (31 mg, 0.083 mmol) and the reaction was heated to 125° C. in a microwave reactor for 20 minutes. The reaction was cooled and partitioned between EtOAc (10 mL) and water (10 mL). The organic phase was washed with water (2×10 mL) and brine (10 mL). The combined organics were dried over magnesium sulfate, filtered, and concentrated. The crude mixture was purified via normal phase column chromatography (silica, 0% to 25% MeOH in EtOAc) followed by reverse phase chromatography (C-18, 5% to 70% ACN in water, 0.1% TFA buffer) to afford 6-3 as a foamy solid after free-basing with saturated NaHCO₃. MS m/z (M+H) 449.1482 found, 449.1487 required.

### EXAMPLE 7

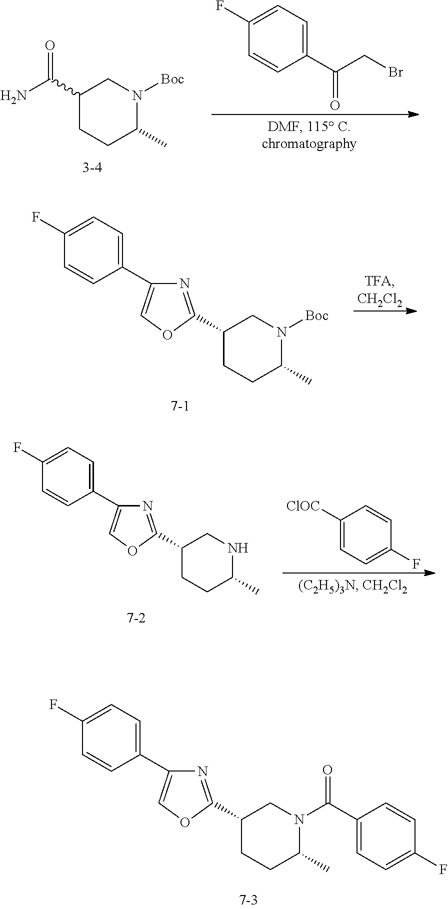

### Methyl (2R,5S)-5-[4-(4-fluorophenyl)-1,3-oxazol-2-yl]-2-methylpiperidine-1-carboxylate (7-1)

A solution of compound 3-4 (320 mg, 1.32 mmol), and 4-fluorophenacyl bromide (573 mg, 2.64 mmol) was heated in DMF (3 mL) at 115° C. for 1 h. The reaction was chromatographed by reverse phase HPLC (C-18, 100×30 mm column, gradient elution with 5% to 95% CH₃CN in H₂O (0.1% TFA) and collecting the second peak to give the title compound.

### (2R,5S)-5-[4-(4-Fluorophenyl)-1,3-oxazol-2-yl]-2-methylpiperidine (7-2)

TFA (1 mL) was added to a solution of methyl (2R,5S)-5-[4-(4-fluorophenyl)-1,3-oxazol-2-yl]-2-methylpiperidine-1-carboxylate (7-1) (mg, mmol) in CH₂Cl₂ (2 mL) and stirred at room temperature for 1 h. (1.0 g, 17 mmol). The solution was washed with NaHCO₃, saturated NaCl solution and dried over Na₂SO₄. Filtration and concentration in vacuo gave the title compound.

### (4-Fluorophenyl){(2R,5S)-5-[4-(4-fluorophenyl)-1,3-oxazol-2-yl]-2-methylpiperidin-1-yl}methanone (7-3)

A solution of 4-fluorophenyl){(2R,5S)-5-[4-(4-fluorophenyl)-1,3-oxazol-2-yl]-2-methylpiperidin-1-yl}methanone (7-2) (21.7 mg, 0.084 mmol) and triethylamine (0.034 mL, 0.251 mmol) in CH₂Cl₂ (2 mL) was cooled to 0° C. A solution of 4-fluorobenzoyl chloride (0.012 mL, 0.100 mmol) in CH₂Cl₂ (0.5 mL) was added and solution warmed to room temperature and stirred 30 min. The reaction solution was concentrated in vacuo and the crude product dissolved in CH₃CN and chromatographed by reverse phase chromatography (C-18, 0 to 95% CH₃CN in water, 0.1% TFA buffer) to give the title compound. MS m/z (M+H) 383.1570 found, 383.1566 required.

### EXAMPLE 8

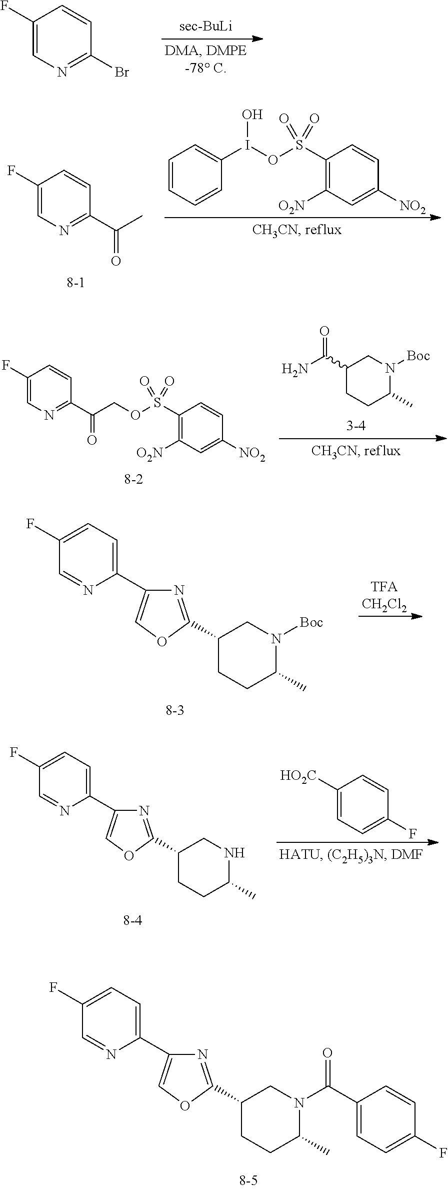

### 2-Acetyl-5-fluoropyridine (8-1)

A solution of 2-bromo-5-fluoropyridine (5.64 g, 32.0 mmol) in DMPE (100 mL) was cooled to −78° C. and sec-butyllithium added (23.35 mL, 32.7 mmol) over a period of 30 min. The reaction was stirred for 2 h and dimethylacetamide was added (3.34 mL, 35.9 mmol) in DMPE (50 mL) over 15 min. The reaction was stirred an additional 15 min then quenched with ice-water (10 mL) and the two phases separated. The organic phase was separated and washed with saturated brine and concentrated to give the crude product. The crude product was purified by silica gel column chromatography (120 g column, eluting with 0 to 5% methanol in CH₂Cl₂) to give the title compound.

### 2-(5-Fluoropyridin-2-yl)-2-oxoethyl 2,4-dinitrobenzenesulfonate (8-2)

A solution of 8-1 (950 mg, 6.83 mmol) and {[(2,4-dinitrophenyl)sulfonyl]oxy}(hydroxy)phenyl-λ³-iodane (3.83 g, 8.19 mmol) (prepared according to the procedure described in Y. Yamamoto et al, Synlett. 2005, 16, 2466-2488) in CH₃CN (30 mL) was refluxed for 2 h. The solvent was removed in vacuo and the crude product dissolved in CH₂Cl₂ and CH₃CN. The crude product was purified by silica gel column chromatography (120 g column, eluting with 10 to 30% EtOAc in hexane) to give the title compound.

### tert-Butyl (2R,5S)-5-[4-(5-fluoropyridin-2-yl)-1,3-oxazol-2-yl]-2-methylpiperidine-1-carboxylate (8-3)

A mixture of 8-2 (1.019 g, 2.64 mmol) and 3-4 (534 mg, 2.20 mmol) was refluxed in CH₃CN (40 mL) overnight under nitrogen, according to the procedure described in J. C. Lee et al., Syn. Comm., 2003, 33(9) 1611-1614. The solvent was removed in vacuo and the crude product was purified by silica gel column chromatography (40 g column, eluting with 0 to 30% EtOAc in hexane) to give the title compound.

### 5-Fluoro-2-{2-[(3S,6R)-6-methylpiperidin-3-yl]-1,3-oxazol-4-yl{pyridine (8-4)

TFA (2 mL) was added to a solution of 8-3 (93 mg, 0.257 mmol) in CH₂Cl₂ (10 mL) and stirred at room temperature overnight. The solution was washed with NaHCO₃, saturated NaCl solution and dried over Na₂SO₄. The crude product was purified by silica gel column chromatography (12 g column, eluting with 0 to 10% (1% NH₄OH in methanol) in CH₂Cl₂) to give the title compound.

### (4-Fluorophenyl){(2R,5S)-5-[4-(5-fluoropyridin-2-yl)-1,3-oxazol-2-yl]-2-methylpiperidine-1-yl}methanone (8-5)

To a solution of 4-fluorobenzoic acid (8.04 mg, 0.057 mmol) and triethylamine (0.0109 mL, 0.080 mmol) in DMF (1 mL) was added HATU (16 mg, 0.042 mmol) and 8-4 (10 mg, 0.038 mmol). The reaction was stirred for 5 min, and then purified by reverse phase HPLC to obtain the title compound. MS m/z (M+H) 384.1518 found, 384.1518 required.

The following compounds were prepared using the foregoing methodology, but substituting the appropriately substituted reagent, as described in the foregoing Reaction Schemes and Examples. The requisite starting materials were commercially available, described in the literature or readily synthesized by one skilled in the art of organic synthesis without undue experimentation.

| Cmpd | Structure | Name | MS m/z (M + H) |
|---|---|---|---|
| 9-1 | 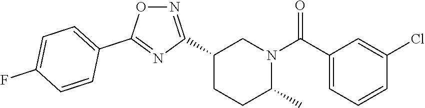 | (2R,5S)-1-(3- chlorobenzoyl)-5-[5-(4- fluorophenyl)-1,2,4- oxadiazol-3-yl]-2- methylpiperidine | 400.1233 found, 400.1150 required. |
| 9-2 | 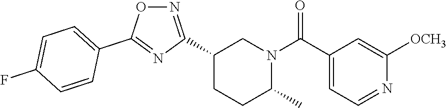 | 4-({(2R,5S)-5-[5-(4- fluorophenyl)-1,2,4- oxadiazol-3-yl]-2- methylpiperidin-1- yl}carbonyl)-2- methoxypyridine | 397.1664 found, 396.1598 required. |
| 9-3 | 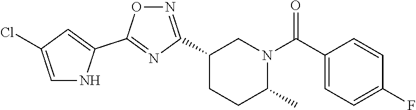 | (2R,5S)-5-[5-(4-chloro-1H- pyrrol-2-yl)-1,2,4- oxadiazol-3-yl]-1-(4- fluorobenzoyl)-2- methylpiperidine | 389.1182 found, 389.1102 required. |
| 9-4 | 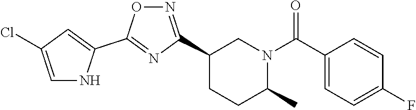 | (2R,5S)-5-[5-(4-chloro-1H- pyrrol-2-yl)-1,2,4- oxadiazol-3-yl]-1-(4- fluorobenzoyl)-2- methylpiperidine | 389.1186 found, 389.1102 required. |
| 9-5 | 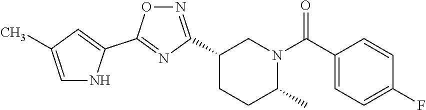 | (2R,5S)-1-(4- fluorobenzoyl)-2-methyl-5- [5-(4-methyl-1H-pyrrol-2- yl)-1,2,4-oxadiazol-3- yl]piperidine | 369.1715 found, 369.1649 required. |
| 9-6 | 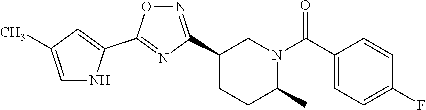 | (2S,5R)-1-(4- fluorobenzoyl)-2-methyl-5- [5-(4-methyl-1H-pyrrol-2- yl)-1,2,4-oxadiazol-3- yl]piperidine | 369.1713 found, 369.1649 required. |
| 9-7 | 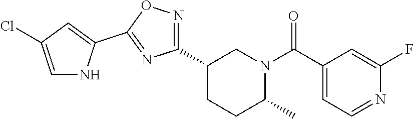 | 4-({(2R,5S)-5-[5-(4-chloro- 1H-pyrrol-2-yl)-1,2,4- oxadiazol-3-yl]-2- methylpiperidin-1- yl}carbonyl)-2- fluoropyridine | 390.1130 found, 390.1055 required. |
| 9-8 | 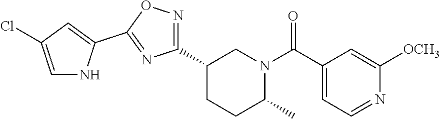 | 4-({(2R,5S)-5-[5-(4-chloro- 1H-pyrrol-2-yl)-1,2,4- oxadiazol-3-yl]-2- methylpiperidin-1- yl}carbonyl)-2- methoxypyridine | 402.00 found, 402.12 required. |
| 9-9 | 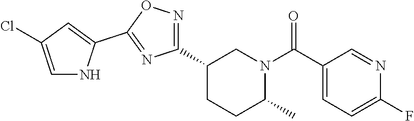 | 5-({(2R,5S)-5-[5-(4-chloro- 1H-pyrrol-2-yl)-1,2,4- oxadiazol-3-yl]-2- methylpiperidin-1- yl}carbonyl)-2- fluoropyridine | 390.1133 found, 390.1055 required. |
| 9-10 | 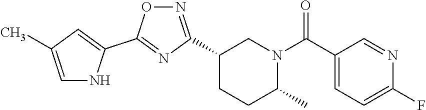 | 2-fluoro-5-({(2R,5S)-2- methyl-5-[5-(4-methyl-1H- pyrrol-2-yl)-1,2,4- oxadiazol-3-yl]piperidin-1- yl}carbonyl)pyridine | 370.00 found, 370.39 required. |
| 9-11 | 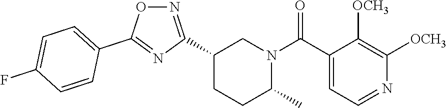 | 4-({(2R,5S)-5-[5-(4- fluorophenyl)-1,2,4- oxadiazol-3-yl]-2- methylpiperidin-1- yl}carbonyl)-2,3- dimethoxypyridine | 415.1589 found, 415.1503 required. |
| 9-12 | 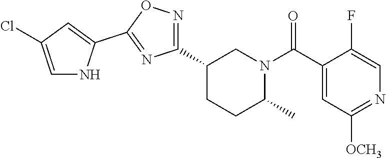 | 4-({(2R,5S)-5-[5-(4-chloro- 1H-pyrrol-2-yl)-1,2,4- oxadiazol-3-yl]-2- methylpiperidin-1- yl}carbonyl)-5-fluoro-2- methoxypyridine | 420.1245 found, 420.1160 required. |
| 9-13 | 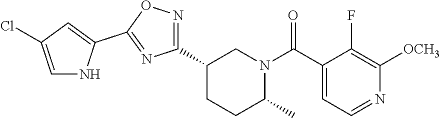 | 4-({(2R,5S)-5-[5-(4-chloro- 1H-pyrrol-2-yl)-1,2,4- oxadiazol-3-yl]-2- methylpiperidin-1- yl}carbonyl)-3-fluoro-2- methoxypyridine | 420.1245 found, 420.1160 required. |
| 9-14 | 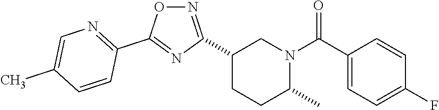 | 2-{3-[(3,6-cis)-1-(4- fluorobenzoyl)-6- methylpiperidin-3-yl]-1,2,4- oxadiazol-5-yl}-5- methylpyridine | 381.1727 found, 381.1649 required. |
| 9-15 | 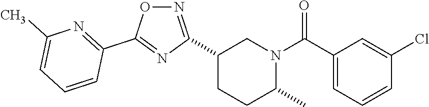 | 2-{3-[(3,6-cis)-1-(3- chlorobenzoyl)-6- methylpiperidin-3-yl]-1,2,4- oxadiazol-5-yl}-6- methylpyridine | 397.1418 found, 397.1353 required. |
| 9-16 | 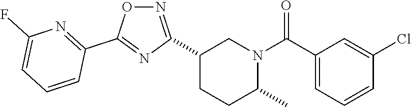 | 2-{3-[(3,6-cis)-1-(3- chlorobenzoyl)-6- methylpiperidin-3-yl]-1,2,4- oxadiazol-5-yl}-6- fluoropyridine | 385.1469 found, 385.1398 required. |
| 9-17 | 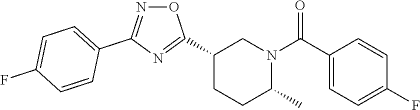 | (2R,5S)-1-(4- fluorobenzoyl)-5-[3-(4- fluorophenyl)-1,2,4- oxadiazol-5-yl]-2- methylpiperidine | 384.1519 found, 384.1445 required. |
| 9-18 | 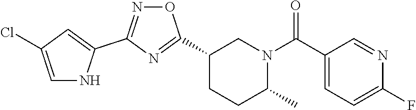 | 5-({(2R,5S)-5-[3-(4-chloro- 1H-pyrrol-2-yl)-1,2,4- oxadiazol-5-yl]-2- methylpiperidin-1- yl}carbonyl)-2- fluoropyridine | 390.1137 found, 390.1055 required. |
| 9-19 | 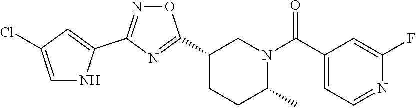 | 4-({(2R,5S)-5-[3-(4-chloro- 1H-pyrrol-2-yl)-1,2,4- oxadiazol-5-yl]-2- methylpiperidin-1- yl}carbonyl)-2- fluoropyridine | 390.1135 found, 390.1055 required. |
| 9-20 | 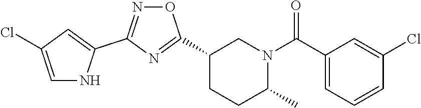 | (2R,5S)-1-(3- chlorobenzoyl)-5-[3-(4- chloro-1H-pyrrol-2-yl)- 1,2,4-oxadiazol-5-yl]-2- methylpiperidine | 405.0896 found, 404.0807 required. |
| 9-21 | 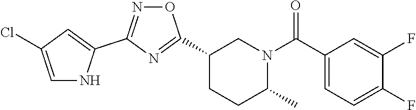 | (2R,5S)-5-[3-(4-chloro-1H- pyrrol-2-yl)-1,2,4- oxadiazol-5-yl]-1-(3,4- difluorobenzoyl)-2- methylpiperidine | 407.1087 found, 407.1008 required. |
| 9-22 | 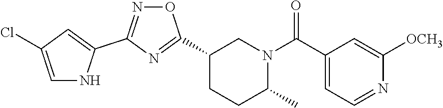 | 4-({(2R,5S)-5-[3-(4-chloro- 1H-pyrrol-2-yl)-1,2,4- oxadiazol-5-yl]-2- methylpiperidin-1- yl}carbonyl)-2- methoxypyridine | 402.1328 found, 402.1255 required. |
| 9-23 | 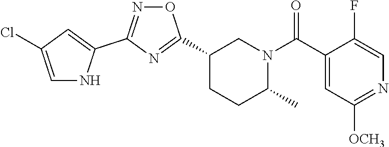 | 4-({(2R,5S)-5-[3-(4-chloro- 1H-pyrrol-2-yl)-1,2,4- oxadiazol-5-yl]-2- methylpiperidin-1- yl}carbonyl)-5-fluoro-2- methoxypyridine | 420.1238 found, 420.1160 required. |
| 9-24 | 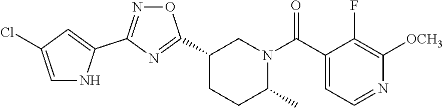 | 4-({(2R,5S)-5-[3-(4-chloro- 1H-pyrrol-2-yl)-1,2,4- oxadiazol-5-yl]-2- methylpiperidin-1- yl}carbonyl)-3-fluoro-2- methoxypyridine | 420.1244 found, 420.1160 required. |
| 9-25 | 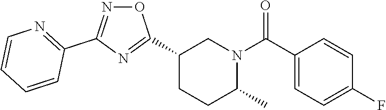 | 2-{5-[(3S,6R)-1-(4- fluorobenzoyl)-6- methylpiperidin-3-yl]-1,2,4- oxadiazol-3-yl}pyridine | 367.1562 found, 367.1492 required. |
| 9-26 | 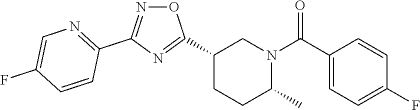 | 5-fluoro-2-{5-[(3S,6R)-1- (4-fluorobenzoyl)-6- methylpiperidin-3-yl]-1,2,4- oxadiazol-3-yl}pyridine | 385.1473 found, 385.1398 required. |
| 9-27 | 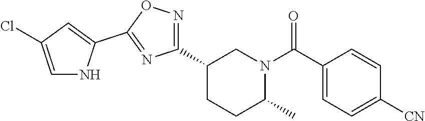 | 4-({(2R,5S)-5-[5-(4-chloro- 1H-pyrrol-2-yl)-1,2,4- oxadiazol-3-yl]-2- methylpiperidin-1- yl}carbonyl)benzonitrile | 396.1210 found, 396.1222 required. |
| 9-28 | 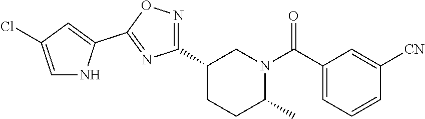 | 3-({(2R,5S)-5-[5-(4-chloro- 1H-pyrrol-2-yl)-1,2,4- oxadiazol-3-yl]-2- methylpiperidin-1- yl}carbonyl)benzonitrile | 396.1228 found, 396.1222 required. |
| 9-29 | 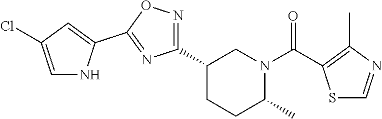 | {(2R,5S)-5-[5-(chloro-1H- pyrrol-2-yl)-1,2,4- oxadiazol-3-yl]-2- methylpiperidin-1-yl}(4- methyl-1,3-thiazol-5- yl)methanone | 392.0937 found, 392.0943 required. |
| 9-30 | 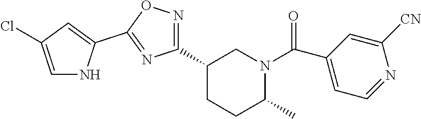 | 4-({(2R,5S)-5-[5-(4-chloro- 1H-pyrrol-2-yl)-1,2,4- oxadiazol-3-yl]-2- methylpiperidin-1- yl}carbonyl)pyridine-2- carbonitrile | 397.1168 found, 397.1174 required. |
| 9-31 | 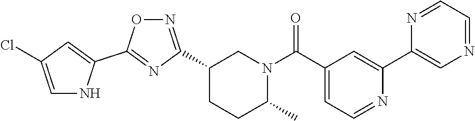 | {(2R,5S)-5-[5-(4-chloro-1H- pyrrol-2-yl)-1,2,4- oxadiazol-3-yl]-2- methylpiperidin-1-yl}[2- (pyrazin-2-yl)pyridin-4- yl]methanone | 450.1438 found, 450.1440 required. |
| 9-32 | 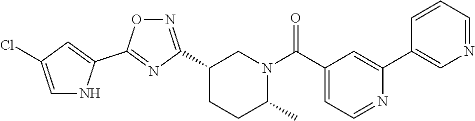 | 2,3′-bipyridin-4-yl{(2R,5S)- 5-[5-(4-chloro-1H-pyrrol-2- yl)-1,2,4-oxadiazol-3-yl]-2- methylpiperidin-1- yl}methanone | 449.1475 found, 449.1487 required. |
| 9-33 | 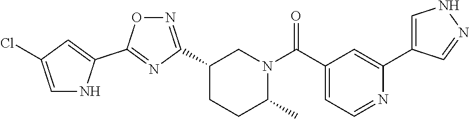 | {(2R,5S)-5-[5-(4-chloro-1H- pyrrol-2-yl)-1,2,4- oxadiazol-3-yl]-2- methylpiperidin-1-yl}[2- (1H-pyrazol-4-yl)pyridin-4- yl]methanone | 438.1433 found, 438.1440 required. |
| 9-34 | 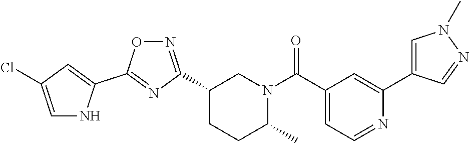 | {(2R,5S)-5-[5-(4-chloro-1H- pyrrol-2-yl)-1,2,4- oxadiazol-3-yl]-2- methylpiperidin-1-yl}[2-(1- methyl-1H-pyrazol-4- yl)pyridin-4-yl]methanone | 452.1588 found, 452.1596 required. |
| 9-35 | 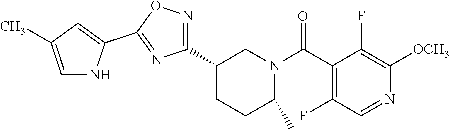 | (3,5-difluoro-2- methoxypyridin-4- yl){(2R,5S)-2-methyl-5-[5- (4-methyl-1H-pyrrol-2-yl)- 1,2,4-oxadiazaol-3- yl]piperidin-1- yl}methanone | 418.5217 found, 418.5217 required. |
| 9-36 | 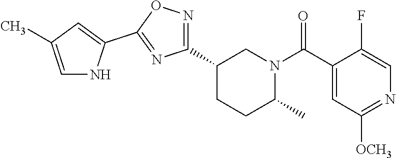 | (5-fluoro-2- methoxypyridin-4- yl){(2R,5S)-2-methyl-5-[5- (4-methyl-1H-pyrrol-2-yl)- 1,2,4-oxadiazol-3- yl]piperidin-1- yl}methanone | 400.1779 found, 400.1779 required. |
| 9-37 | 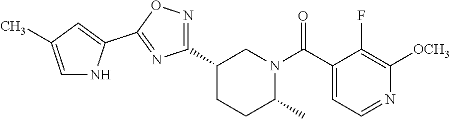 | (3-fluoro-2- methoxypyridin-4- yl){(2R,5S)-2-methyl-5-[5- (4-methyl-1H-pyrrol-2-yl)- 1,2,4-oxadiazol-3- yl]piperidin-1- yl}methanone | 400.1779 found, 400.1779 required. |
| 9-38 | 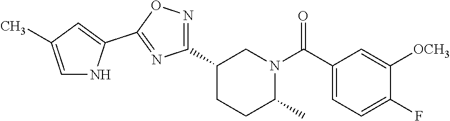 | (4-fluoro-3- methoxyphenyl){(2R,5S)-2- methyl-5-[5-(4-methyl-1H- pyrrol-2-yl)-1,2,4- oxadiazol-3-yl]piperidin-1- yl}methanone | 399.1830 found, 399.1827 required. |
| 9-39 | 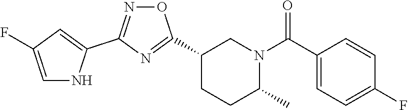 | (4-fluorophenyl){(2R,5S)-5- [3-(4-fluoro-1H-pyrrol-2- yl)-1,2,4-oxadiazol-5-yl]-2- methylpiperidin-1- yl}methanone | 373.1478 found, 373.1471 required. |
| 9-40 | 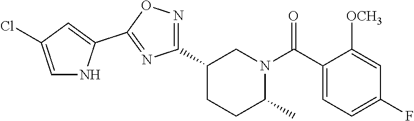 | {(2R,5S)-5-[5-(4-chloro- 1H-pyrrol-2-yl)-1,2,4- oxadiazol-3-yl]-2- methylpiperidin-1-yl}(4- fluoro-2- methoxyphenyl)methanone | 419.1291 found, 419.1281 required. |
| 9-41 | 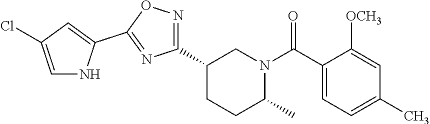 | {(2R,5S)-5-[5-(4-chloro- 1H-pyrrol-2-yl)-1,2,4- oxaidazol-3-yl]-2- methylpiperidin-1-yl}(2- methoxy-4- methylphenyl)methanone | 415.1553 found, 415 1532 required. |
| 9-42 | 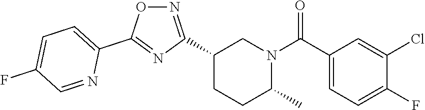 | (3-chloro-4- fluorophenyl){(2R,5S)-5-[5- (5-fluoropyridin-2-yl)-1,2,4- oxadiazol-3-yl]-2- methylpiperidin-1- yl}methanone | 419.1083 found, 419.1081 required. |
| 9-43 | 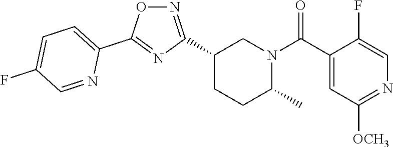 | (5-fluoro-2- methoxypyridin-4- yl){(2R,5S)-5-[5-(5- fluoropyridin-2-yl)-1,2,4- oxadiazol-3-yl]-2- methylpiepridin-1- yl}methanone | 416.1529 found, 416.1529 required. |
| 9-44 | 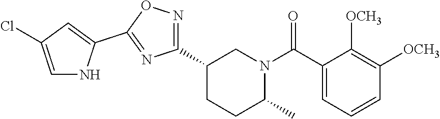 | {(2R,5S)-5-[5-(4-chloro- 1H-pyrrol-2-yl)-1,2,4- oxadiazol-3-yl]-2- methylpiperidin-1-yl}(2,3- dimethoxyphenyl)methanone | 431.1504 found, 431.1481 required. |
| 9-45 | 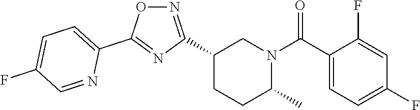 | (2,4- difluorophenyl){(2R,5S)-5- [5-(5-fluoropyridin-2-yl)- 1,2,4-oxadiazol-3-yl]-2- methylpiepridin-1- yl}methanone | 403.1374 found, 403.1376 required. |
| 9-46 | 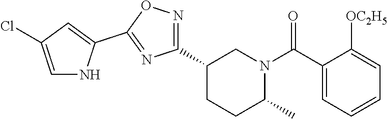 | {(2R,5S)-5-[5-(4-chloro- 1H-pyrrol-2-yl)-1,2,4- oxadiazol-3-yl]-2- methylpiperidin-1-yl}(2- ethoxyphenyl)methanone | 415.1535 found, 415.1532 required. |
| 9-47 | 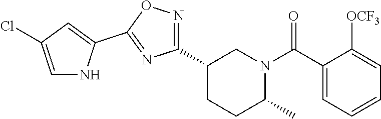 | {(2R,5S)-5-[5-(4-chloro- 1H-pyrrol-2-yl)1,2,4- oxadiazol-3-yl]-2- methylpiperidin-1-yl}[2- (trifluoromethoxy)phenyl] methanone | 455.1092 found, 455.1093 required. |
| 9-48 | 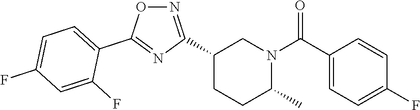 | {(2R,5S)-5-[5-(2,4- difluorophenyl)-1,2,4- oxadiazol-3-yl]-2- methylpiperidin-1-yl}(4- fluorophenyl)methanone | 402.1425 found, 402.1424 required. |
| 9-49 | 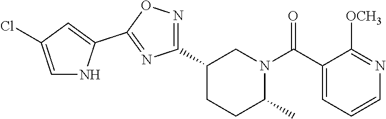 | {(2R,5S)-5-[5-(4-chloro- 1H-pyrrol-2-yl)-1,2,4- oxadiazol-3-yl]-2- methylpiperidin-1-yl}(2- methoxypyridin-3- yl)methanone | 402.1348 found, 402.1328 required. |
| 9-50 | 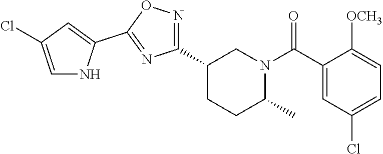 | (5-chloro-2- methoxyphenyl){(2R,5S)-5- [5-(4-chloro-1H-pyrrol-2- yl)-1,2,4-oxadiazol-3-yl]-2- methylpiperidin-1- yl}methanone | 435.1002 found, 435.0985 required. |
| 9-51 | 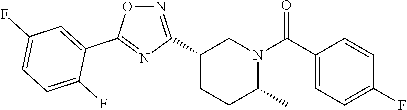 | {(2R,5S)-5-[5-(2,5- difluorophenyl)-1,2,4- oxadiazol-3-yl]-2- methylpiperidin-1-yl}(4- fluorophenyl)methanone | 402.1424 found, 402.1424 required. |
| 9-52 | 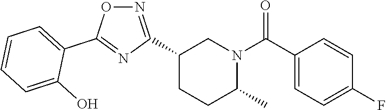 | (4-fluorophenyl){(2R,5S)-5- [5-(2-hydroxyphenyl)-1,2,4- oxadiazol-3-yl]-2- methylpiperidin-1- yl}methanone | 382.1561 found, 382.1562 required. |
| 9-53 | 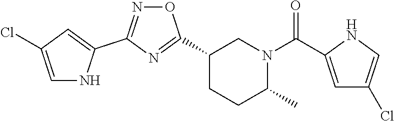 | (4-chloro-1H-pyrrol-2- yl){(2R,5S)-5-[3-(4-chloro- 1H-pyrrol-2-yl)-1,2,4- oxadiazol-5-yl]-2- methylpiperidin-1- yl}methanone | 394.0832 found, 394.0832 required. |
| 9-54 | 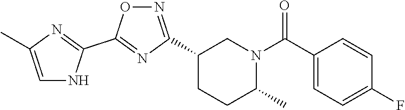 | (4-fluorophenyl)-{(2R,5S)- 2-methyl-5-[5-(4-methyl- 1H-imidazol-2-yl)-1,2,4- oxadiazol-3-yl]piperidin-1- yl}methanone | 370.1669 found, 370.1674 required. |

While the invention has been described and illustrated with reference to certain particular embodiments thereof, those skilled in the art will appreciate that various adaptations, changes, modifications, substitutions, deletions, or additions of procedures and protocols may be made without departing from the spirit and scope of the invention.

1. A compound of the formula I: 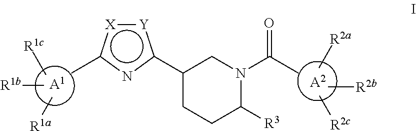
wherein:
A¹ is selected from the group consisting of phenyl, naphthyl and heteroaryl;
A² is selected from the group consisting of phenyl, naphthyl and heteroaryl;
X is selected from N, O and C(R¹³),
Y is selected from N and O, wherein X is N and Y is O, to form a oxadiazole ring, or X is O and Y is N, to form a oxadiazole ring, or X is C(R¹³) and Y is O to form an oxazole ring;
R^{1a}, R^{1b} and R^{1c} may be absent if the valency of A¹ does not permit such substitution and are independently selected from the group consisting of:
(1) hydrogen,
(2) halogen,
(3) hydroxyl,
(4) —(C═O)ₘ—Oₙ—C₁₋₆alkyl, where m is 0 or 1, n is 0 or 1 (wherein if m is 0 or n is 0, a bond is present) and where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heterocycle, where the heterocycle is unsubstituted or substituted with one or more substituents selected from R¹³,
(10) —(C═O)ₘ—NR¹⁰R¹¹, wherein R¹⁰ and R¹¹ are independently selected from the group consisting of:
(a) hydrogen,
(b) C₁₋₆alkyl, which is unsubstituted or substituted with R¹³,
(c) C₃₋₆alkenyl, which is unsubstituted or substituted with R¹³,
(d) C₃₋₆alkynyl, which is unsubstituted or substituted with R¹³,
(e) C₃₋₆cycloalkyl which is unsubstituted or substituted with R¹³,
(f) phenyl, which is unsubstituted or substituted with R¹³, and
(g) heterocycle, which is unsubstituted or substituted with R¹³,
(11) —S(O)₂—NR¹⁰R¹¹,
(12) —S(O)_{q}—R¹², where q is 0, 1 or 2 and where R¹² is selected from the definitions of R¹⁰ and R¹¹,
(13) —CO₂H,
(14) —CN, and
(15) —NO₂;
R^{2a}, R^{2b} and R^{2c} may be absent if the valency of A² does not permit such substitution and are independently selected from the group consisting of:
(1) hydrogen,
(2) halogen,
(3) hydroxyl,
(4) —(C═O)ₘ—Oₙ—C₁₋₆alkyl, where the alkyl is unsubstituted or substituted with one or more substituents selected from R¹³,
(5) —(C═O)ₘ—Oₙ—C₃₋₆cycloalkyl, where the cycloalkyl is unsubstituted or substituted with one or more substituents selected from R¹³,
(6) —(C═O)ₘ—C₂₋₄alkenyl, where the alkenyl is unsubstituted or substituted with one or more substituents selected from R¹³,
(7) —(C═O)ₘ—C₂₋₄alkynyl, where the alkynyl is unsubstituted or substituted with one or more substituents selected from R¹³,
(8) —(C═O)ₘ—Oₙ-phenyl or —(C═O)ₘ—Oₙ-napthyl, where the phenyl or naphthyl is unsubstituted or substituted with one or more substituents selected from R¹³,
(9) —(C═O)ₘ—Oₙ-heterocycle, where the heterocycle is unsubstituted or substituted with one or more substituents selected from R¹³,
(10) —(C═O)ₘ—NR¹⁰R¹¹,
(11) —S(O)₂—NR¹⁰R¹¹,
(12) —S(O)_{q}—R¹²,
(13) —CO²H,
(14) —CN, and
(15) —NO₂;
R³ is C₁₋₆alkyl;
R¹³ is selected from the group consisting of:
(1) halogen,
(2) hydroxyl,
(3) —(C═O)ₘ—Oₙ—C₁₋₆alkyl, where the alkyl is unsubstituted or substituted with one or more substituents selected from R¹⁴,
(4) —Oₙ—(C₁₋₃)perfluoroalkyl,
(5) —(C═O)ₘ—Oₙ—C₃₋₆cycloalkyl, where the cycloalkyl is unsubstituted or substituted with one or more substituents selected from R¹⁴,
(6) —(C═O)ₘ—C₂₋₄alkenyl, where the alkenyl is unsubstituted or substituted with one or more substituents selected from R¹⁴,
(7) —(C═O)ₘ—C₂₋₄alkynyl, where the alkynyl is unsubstituted or substituted with one or more substituents selected from R¹⁴,
(8) —(C═O)ₘ—Oₙ-phenyl or —(C═O)ₘ—Oₙ-napthyl, where the phenyl or naphthyl is unsubstituted or substituted with one or more substituents selected from R¹⁴,
(9) —(C═O)ₘ—Oₙ-heterocycle, where the heterocycle is unsubstituted or substituted with one or more substituents selected from R¹⁴,
(10) —(C═O)ₘ—NR¹⁰R¹¹,
(11) —S(O)₂—NR¹⁰R¹¹,
(12) —S(O)_{q}—R¹²,
(13) —CO₂H,
(14) —CN, and
(15) —NO₂;
R¹⁴ is selected from the group consisting of:
(1) hydroxyl,
(2) halogen,
(3) C₁₋₆alkyl,
(4) —C₃₋₆cycloalkyl,
(5) —O—C₁₋₆alkyl,
(6) —O(C═O)—C₁₋₆alkyl,
(7) —NH—C₁₋₆alkyl,
(8) phenyl,
(9) heterocycle,
(10) —CO₂H, and
(11) —CN;
or a pharmaceutically acceptable salt thereof.

2. The compound of claim 1 of the formula Ia: 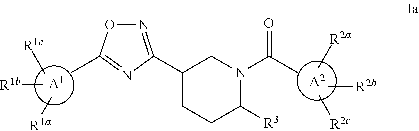
or a pharmaceutically acceptable salt thereof.

3. The compound of claim 1 of the formula Ib: 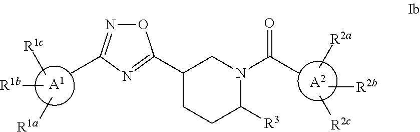
or a pharmaceutically acceptable salt thereof.

4. The compound of claim 1 of the formula Ic: 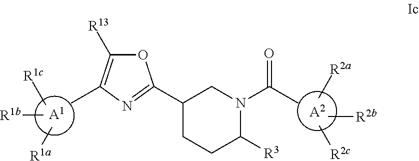
pharmaceutically acceptable salt thereof.

5. The compound of claim 1 wherein A¹ is selected from the group consisting of: phenyl, pyridyl and pyrrolyl.

6. The compound of claim 1 wherein A² is selected from the group consisting of: phenyl and pyridyl.

7. The compound of claim 1 wherein R^{1a}, R^{1b} and R^{1c} are independently selected from the group consisting of:
(1) hydrogen,
(2) halogen,
(3) hydroxyl,
(4) C₁₋₆alkyl, which is unsubstituted or substituted with halogen, hydroxyl, phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, —O—C₁₋₆alkyl or —NO₂.

8. The compound of claim 1 wherein A¹ is phenyl, pyridyl or pyrrolyl and R^{1a}, R^{1b} and R^{1c} are independently selected from the group consisting of:
(1) hydrogen,
(2) chloro,
(3) fluroro, and
(4) methyl.

9. The compound of claim 1 wherein R^{2a}, R^{2c} and R^{2c} are independently selected from the group consisting of:
(1) hydrogen,
(2) halogen,
(3) hydroxyl,
(4) C₁₋₆alkyl, which is unsubstituted or substituted with halogen, hydroxyl or phenyl or napthyl,
(5) —O—C₁₋₆alkyl, which is unsubstituted or substituted with halogen, hydroxyl or phenyl,
(6) heteroaryl, wherein heteroaryl is selected from pyrrolyl, imidazolyl, indolyl, pyridyl, and pyrimidinyl, which is unsubstituted or substituted with halogen, hydroxyl, C₁₋₆alkyl, —O—C₁₋₆alkyl or —NO₂,
(7) phenyl, which is unsubstituted or substituted with halogen, hydroxyl, C₁₋₆alkyl, —O—C₁₋₆alkyl or —NO₂,
(8) —O-phenyl, which is unsubstituted or substituted with halogen, hydroxyl, C₁₋₆alkyl, —O—C₁₋₆alkyl or —NO₂, and
(9) —NH—C₁₋₆alkyl, or —N(C₁₋₆alkyl)(C₁₋₆alkyl), which is unsubstituted or substituted with halogen, hydroxyl, C₁₋₆alkyl, —O—C₁₋₆alkyl or —NO₂.

10. The compound of claim 1 wherein A² is phenyl or pyridyl and R^{2a}, R^{2b} and R^{2c} are independently selected from the group consisting of:
(1) hydrogen,
(2) chloro,
(3) fluoro,
(4) bromo,
(5) methoxy,
(6) t-butoxy,
(7) difluoromethyl, and
(8) trifluoromethyl.

11. The compound of claim 1 wherein R³ is methyl.

12. A compound which is selected from the group consisting of:
(2S,5R)-1-(4-fluorobenzoyl)-5-[5-(4-fluorophenyl)-1,2,4-oxadiazol-3-yl]-2-methylpiperidine;
(2R,5S)-1-(4-fluorobenzoyl)-5-[5-(4-fluorophenyl)-1,2,4-oxadiazol-3-yl]-2-methylpiperidine;
(2S,5R)-5-[5-(4-chloro-1H-pyrrol-2-yl)-1,2,4-oxadiazol-3-yl]-1-(4-fluorobenzoyl)-2-methylpiperidine;
(2R,5S)-5-[5-(4-chloro-1H-pyrrol-2-yl)-1,2,4-oxadiazol-3-yl]-1-(4-fluorobenzoyl)-2-methylpiperidine;
5-fluoro-2-{3-[(3S,6R)-1-(4-fluorobenzoyl)-6-methylpiperidin-3-yl]-1,2,4-oxadiazol-5-yl}pyridine;
(2R,5S)-5-[3-(4-chloro-1H-pyrrol-2-yl)-1,2,4-oxadiazol-5-yl]-1-(4-fluorobenzoyl)-2-methylpiperidine;
2,4′-bipyridin-4-yl{(2R,5S)-5-[5-(4-chloro-1H-pyrrol-2-yl)-1,2,4-oxadiazol-3-yl]-2-methylpiperidin-1-yl}methanone;
2,2′-bipyridin-4-yl{(2R,5S)-5-[5-(4-chloro-1H-pyrrol-2-yl)-1,2,4-oxadiazol-3-yl]-2-methylpiperidin-1-yl}methanone;
(4-fluorophenyl){(2R,5S)-5-[4-(4-fluorophenyl)-1,3-oxazol-2-yl]-2-methylpiperidin-1-yl}methanone;
(4-fluorophenyl){(2R,5S)-5-[4-(5-fluoropyridin-2-yl)-1,3-oxazol-2-yl]-2-methylpiperidin-1-yl}methanone;
(2R,5S)-1-(3-chlorobenzoyl)-5-[5-(4-fluorophenyl)-1,2,4-oxadiazol-3-yl]-2-methylpiperidine;
4-({(2R,5S)-5-[5-(4-fluorophenyl)-1,2,4-oxadiazol-3-yl]-2-methylpiperidin-1-yl}carbonyl)-2-methoxypyridine;
(2R,5S)-5-[5-(4-chloro-1H-pyrrol-2-yl)-1,2,4-oxadiazol-3-yl]-1-(4-fluorobenzoyl)-2-methylpiperidine;
(2R,5S)-5-[5-(4-chloro-1H-pyrrol-2-yl)-1,2,4-oxadiazol-3-yl]-1-(4-fluorobenzoyl)-2-methylpiperidine;
(2R,5S)-1-(4-fluorobenzoyl)-2-methyl-5-[5-(4-methyl-1H-pyrrol-2-yl)-1,2,4-oxadiazol-3-yl]piperidine;
(2S,5R)-1-(4-fluorobenzoyl)-2-methyl-5-[5-(4-methyl-1H-pyrrol-2-yl)-1,2,4-oxadiazol-3-yl]piperidine;
4-({(2R,5S)-5-[5-(4-chloro-1H-pyrrol-2-yl)-1,2,4-oxadiazol-3-yl]-2-methylpiperidin-1-yl}carbonyl)-2-fluoropyridine;
4-({(2R,5S)-5-[5-(4-chloro-1H-pyrrol-2-yl)-1,2,4-oxadiazol-3-yl]-2-methylpiperidin-1-yl}carbonyl)-2-methoxypyridine;
5-({(2R,5S)-5-[5-(4-chloro-1H-pyrrol-2-yl)-1,2,4-oxadiazol-3-yl]-2-methylpiperidin-1-yl}carbonyl)-2-fluoropyridine;
2-fluoro-5-({(2R,5S)-2-methyl-5-[5-(4-methyl-1H-pyrrol-2-yl)-1,2,4-oxadiazol-3-yl]piperidin-1-yl}carbonyl)pyridine;
4-({(2R,5S)-5-[5-(4-fluorophenyl)-1,2,4-oxadiazol-3-yl]-2-methylpiperidin-1-yl}carbonyl)-2,3-dimethoxypyridine;
4-({(2R,5S)-5-[5-(4-chloro-1H-pyrrol-2-yl)-1,2,4-oxadiazol-3-yl]-2-methylpiperidin-1-yl}carbonyl)-5-fluoro-2-methoxypyridine;
4-({(2R,5S)-5-[5-(4-chloro-1H-pyrrol-2-yl)-1,2,4-oxadiazol-3-yl]-2-methylpiperidin-1-yl}carbonyl)-3-fluoro-2-methoxypyridine;
2-{3-[(3,6-cis)-1-(4-fluorobenzoyl)-6-methylpiperidin-3-yl]-1,2,4-oxadiazol-5-yl}-5-methylpyridine;
2-{3-[(3,6-cis)-1-(3-chlorobenzoyl)-6-methylpiperidin-3-yl]-1,2,4-oxadiazol-5-yl}-6-methylpyridine;
2-{3-[(3,6-cis)-1-(3-chlorobenzoyl)-6-methylpiperidin-3-yl]-1,2,4-oxadiazol-5-yl}-6-fluoropyridine;
(2R,5S)-1-(4-fluorobenzoyl)-5-[3-(4-fluorophenyl)-1,2,4-oxadiazol-5-yl]-2-methylpiperidine;
5-({(2R,5S)-5-[3-(4-chloro-1H-pyrrol-2-yl)-1,2,4-oxadiazol-5-yl]-2-methylpiperidin-1-yl}carbonyl)-2-fluoropyridine
4-({(2R,5S)-5-[3-(4-chloro-1H-pyrrol-2-yl)-1,2,4-oxadiazol-5-yl]-2-methylpiperidin-1-yl}carbonyl)-2-fluoropyridine;
(2R,5S)-1-(3-chlorobenzoyl)-5-[3-(4-chloro-1H-pyrrol-2-yl)-1,2,4-oxadiazol-5-yl]-2-methylpiperidine;
(2R,5S)-5-[3-(4-chloro-1H-pyrrol-2-yl)-1,2,4-oxadiazol-5-yl]-1-(3,4-difluorobenzoyl)-2-methylpiperidine;
4-({(2R,5S)-5-[3-(4-chloro-1H-pyrrol-2-yl)-1,2,4-oxadiazol-5-yl]-2-methylpiperidin-1-yl}carbonyl)-2-methoxypyridine;
4-({(2R,5S)-5-[3-(4-chloro-1H-pyrrol-2-yl)-1,2,4-oxadiazol-5-yl]-2-methylpiperidin-1-yl}carbonyl)-5-fluoro-2-methoxypyridine;
4-({(2R,5S)-5-[3-(4-chloro-1H-pyrrol-2-yl)-1,2,4-oxadiazol-5-yl]-2-methylpiperidin-1-yl}carbonyl)-3-fluoro-2-methoxypyridine;
2-{5-[(3S,6R)-1-(4-fluorobenzoyl)-6-methylpiperidin-3-yl]-1,2,4-oxadiazol-3-yl}pyridine;
5-fluoro-2-{5-[(3S,6R)-1-(4-fluorobenzoyl)-6-methylpiperidin-3-yl]-1,2,4-oxadiazol-3-yl}pyridine;
4-({(2R,5S)-5-[5-(4-chloro-1H-pyrrol-2-yl)-1,2,4-oxadiazol-3-yl]-2-methylpiperidin-1-yl}carbonyl)benzonitrile;
3-({(2R,5S)-5-[5-(4-chloro-1H-pyrrol-2-yl)-1,2,4-oxadiazol-3-yl]-2-methylpiperidin-1-yl}carbonyl)benzonitrile;
{(2R,5S)-5-[5-(4-chloro-1H-pyrrol-2-yl)-1,2,4-oxadiazol-3-yl]-2-methylpiperidin-1-yl}(4-methyl-1,3-thiazol-5-yl)methanone;
4-({(2R,5S)-5-[5-(4-chloro-1H-pyrrol-2-yl)-1,2,4-oxadiazol-3-yl]-2-methylpiperidin-1-yl}carbonyl)pyridine-2-carbonitrile;
{(2R,5S)-5-[5-(4-chloro-1H-pyrrol-2-yl)-1,2,4-oxadiazol-3-yl]-2-methylpiperidin-1-yl}[2-(pyrazin-2-yl)pyridin-4-yl]methanone;
2,3′-bipyridin-4-yl{(2R,5S)-5-[5-(4-chloro-1H-pyrrol-2-yl)-1,2,4-oxadiazol-3-yl]-2-methylpiperidin-1-yl}methanone;
{(2R,5S)-5-[5-(4-chloro-1H-pyrrol-2-yl)-1,2,4-oxadiazol-3-yl]-2-methylpiperidin-1-yl}[2-(1H-pyrazol-4-yl)pyridin-4-yl]methanone;
{(2R,5S)-5-[5-(4-chloro-1H-pyrrol-2-yl)-1,2,4-oxadiazol-3-yl]-2-methylpiperidin-1-yl}[2-(1-methyl-1H-pyrazol-4-yl)pyridin-4-yl]methanone;
(3,5-difluoro-2-methoxypyridin-4-yl){(2R,5S)-2-methyl-5-[5-(4-methyl-1H-pyrrol-2-yl)-1,2,4-oxadiazol-3-yl]piperidin-1-yl}methanone;
(5-fluoro-2-methoxypyridin-4-yl){(2R,5S)-2-methyl-5-[5-(4-methyl-1H-pyrrol-2-yl)-1,2,4-oxadiazol-3-yl]piperidin-1-yl}methanone;
(3-fluoro-2-methoxypyridin-4-yl){(2R,5S)-2-methyl-5-[5-(4-methyl-1H-pyrrol-2-yl)-1,2,4-oxadiazol-3-yl]piperidin-1-yl}methanone;
(4-fluoro-3-methoxyphenyl){(2R,5S)-2-methyl-5-[5-(4-methyl-1H-pyrrol-2-yl)-1,2,4-oxadiazol-3-yl]piperidin-1-yl}methanone;
(4-fluorophenyl){(2R,5S)-5-[3-(4-fluoro-1H-pyrrol-2-yl)-1,2,4-oxadiazol-5-yl]-2-methylpiperidin-1-yl}methanone;
{(2R,5S)-5-[5-(4-chloro-1H-pyrrol-2-yl)-1,2,4-oxadiazol-3-yl]-2-methylpiperidin-1-yl}(4-fluoro-2-methoxyphenyl)methanone;
{(2R,5S)-5-[5-(4-chloro-1H-pyrrol-2-yl)-1,2,4-oxadiazol-3-yl]-2-methylpiperidin-1-yl}(2-methoxy-4-methylphenyl)methanone;
(3-chloro-4-fluorophenyl){(2R,5S)-5-[5-(5-fluoropyridin-2-yl)-1,2,4-oxadiazol-3-yl]-2-methylpiperidin-1-yl}methanone;
(5-fluoro-2-methoxypyridin-4-yl){(2R,5S)-5-[5-(5-fluoropyridin-2-yl)-1,2,4-oxadiazol-3-yl]-2-methylpiperidin-1-yl}methanone;
{(2R,5S)-5-[5-(4-chloro-1H-pyrrol-2-yl)-1,2,4-oxadiazol-3-yl]-2-methylpiperidin-1-yl}(2,3-dimethoxyphenyl)methanone;
(2,4-difluorophenyl){(2R,5S)-5-[5-(5-fluoropyridin-2-yl)-1,2,4-oxadiazol-3-yl]-2-methylpiperidin-1-yl}methanone;
{(2R,5S)-5-[5-(4-chloro-1H-pyrrol-2-yl)-1,2,4-oxadiazol-3-yl]-2-methylpiperidin-1-yl}(2-ethoxyphenyl)methanone;
{(2R,5S)-5-[5-(4-chloro-1H-pyrrol-2-yl)-1,2,4-oxadiazol-3-yl]-2-methylpiperidin-1-yl}[2-(trifluoromethoxy)phenyl]methanone;
{(2R,5S)-5-[5-(2,4-difluorophenyl)-1,2,4-oxadiazol-3-yl]-2-methylpiperidin-1-yl}(4-fluorophenyl)methanone;
{(2R,5S)-5-[5-(4-chloro-1H-pyrrol-2-yl)-1,2,4-oxadiazol-3-yl]-2-methylpiperidin-1-yl}(2-methoxypyridin-3-yl)methanone;
(5-chloro-2-methoxyphenyl){(2R,5S)-5-[5-(4-chloro-1H-pyrrol-2-yl)-1,2,4-oxadiazol-3-yl]-2-methylpiperidin-1-yl}methanone;
{(2R,5S)-5-[5-(2,5-difluorophenyl)-1,2,4-oxadiazol-3-yl]-2-methylpiperidin-1-yl}(4-fluorophenyl)methanone;
(4-fluorophenyl){(2R,5S)-5-[5-(2-hydroxyphenyl)-1,2,4-oxadiazol-3-yl]-2-methylpiperidin-1-yl}methanone;
(4-chloro-1H-pyrrol-2-yl){(2R,5S)-5-[3-(4-chloro-1H-pyrrol-2-yl)-1,2,4-oxadiazol-5-yl]-2-methylpiperidin-1-yl}methanone; and
(4-fluorophenyl){(2R,5S)-2-methyl-5-[5-(4-methyl-1H-imidazol-2-yl)-1,2,4-oxadiazol-3-yl]piperidin-1-yl}methanone;
or a pharmaceutically acceptable salt thereof.

13. A pharmaceutical composition which comprises a pharmaceutically acceptable carrier and a compound of claim 1 or a pharmaceutically acceptable salt thereof.

14. (canceled)

15. (canceled)

16. A method for treating a neurological or psychiatric disorder associated with glutamate dysfunction in a patient in need thereof comprising administering to the patient a therapeutically effective amount of the compound of claim 1 or a pharmaceutically acceptable salt thereof.

17. A method for treating schizophrenia in a mammalian patient in need thereof which comprises administering to the patient a therapeutically effective amount of a compound of claim 1 or a pharmaceutically acceptable salt thereof.
